(19) United States
(12) Patent Application Publication
(10) Pub. No.: US 2025/0031967 A1
(43) Pub. Date: Jan. 30, 2025

(54) QUANTIFICATION OF FLUID-TISSUE EXCHANGE USING PHASE ALTERNATE LABELING WITH NULL RECOVERY MRI

(71) Applicants: The Johns Hopkins University, Baltimore, MD (US); KENNEDY KRIEGER INSTITUTE, Baltimore, MD (US)

(72) Inventors: Jiadi XU, Baltimore, MD (US); Anna M. LI, Baltimore, MD (US)

(73) Assignee: KENNEDY KRIEGER INSTITUTE F.M. KIRBY RESEARCH CENTER, Baltimore, MD (US)

(21) Appl. No.: 18/714,917

(22) PCT Filed: Nov. 8, 2022
(86) PCT No.: PCT/US2022/049297

§ 371 Date: May 30, 2024

Related U.S. Application Data
(60) Provisional application No. 63/285,012, filed on Dec. 1, 2021.

(51) Int. Cl. A61B 5/00; A61B 5/055; G01R 33/56; G01R 33/563

(52) U.S. Cl. CPC A61B 5/0042; A61B 5/055; A61B 5/4881; G01R 33/5602; G01R 33/5607; G01R 33/56341; G01R 33/56366; A61B 2576/026

(57) ABSTRACT

A system for magnetic resonance imaging of water exchange processes includes a primary magnet to provide a magnetic field over an imaging volume, a magnetic gradient coil to generate a spatial encoding in the magnetic field, a radiofrequency (RF) coil, and a data processor. The RF coil acquires, from the imaging volume, at multiple time points, water magnetic resonance signals including a first subset of signals labeled for a water exchange process and a second subset of signals that are not labeled. The data processor is configured to generate labeled images from the first subset of signals, generate control images corresponding to the set of labeled images from the second subset of signals, and calculate one or more parameters to characterize a water exchange process in the imaging volume between a first water compartment and a second water compartment based on the labeled images and the corresponding control images.

### CROSS-REFERENCE OF RELATED APPLICATION

This application claims priority to U.S. Provisional Application No. 63/285,012, filed Dec. 1, 2021, which is incorporated herein by reference in its entirety.

### STATEMENT REGARDING FEDERALLY SPONSORED RESEARCH OR DEVELOPMENT

This invention was made with government support under grant R01HL149742 awarded by the National Institutes of Health. The government has certain rights in the invention.

### BACKGROUND

### 1. Technical Field

Currently claimed embodiments of this invention relate to magnetic resonance imaging (MRI), and more particularly to MRI of water exchange processes in the brain.

### 2. Discussion of Related Art

Cerebrospinal fluid (CSF) plays an essential role in maintaining the homeostasis of the central nervous system, providing buoyancy to the brain (1), serving as an important route for the removal of a variety of waste products produced by cellular metabolism (2). Its circulation has been the subject of speculation and experiment for more than one hundred years. However, its formation and circulation are still under debate. Conventionally, it is believed that the anterior choroidal arterial blood secretes CSF via choroid plexuses (CP) inside the brain ventricles (80-90%), and CSF flows unidirectionally along subarachnoid spaces to be absorbed into venous sinuses. Some other pieces of evidence supported that CSF continuously exchanges with the interstitial fluid (ISF) in its surrounding brain parenchyma, which depends on hydrostatic and osmotic forces, i.e., the transependymal flow (3-7).

The ependymal cells that separate CSF from parenchyma also play an important role in regulating and producing CSF (8). However, the CSF exchange with the parenchyma and ependymal layers and the correlation with brain function has not been well documented. The CSF exchange process may also impact the recently discovered brain lymphatic system, dubbed the glymphatic system, which suggests that the subarachnoid CSF recirculates through the brain parenchyma, exchanges with the ISF, and then flows back to CSF (9-12). The glymphatic system has drawn intensive attention since its discovery because it appeared to clear off soluble amyloid-beta (Aβ) from the brain parenchyma.

Consequently, it is crucial to develop a clinically useful prognostic tool for measuring the CSF-tissue exchange that can be used to diagnose and evaluate brain diseases. Many non-invasion MRI methods, diffusion-based methods (13,14), and time-of-flight-based MRI methods (15) have been implemented to examine the bulk flow inside the ventricles and glymphatic vessels. However, examining the water exchange between CSF and its surrounding tissues still relies heavily on MRI contrast agents such as the intra-cranial (10,16-19) or intrathecal injection (20-22) of gadolinium-based contrast agents and intravenous D-glucose infusion. (23,24) Contrast agent-based MRI methods are far from ideal for routine and repeated measurements on patients.

The T₁, T₂, and apparent diffusion coefficient (ADC) have been observed to differ significantly between CSF (e.g., T₁=3.6 s, T₂=300 ms at 11.7T; ADC=3 μm²/ms) and ISF (e.g., T₁=1.80 s, T₂=40 ms at 11.7T; ADC=0.7 μm²/ms). (23,25) In principle, T₁, T₂, or ADC can selectively label either CSF or ISF to monitor the water exchange process between CSF and its surrounding brain tissues. Previous strategies have included flow-sensitive alternating inversion recovery arterial spin labeling (ASL) and other strategies to measure the blood-brain barrier water permeability by separating intravascular and extravascular water signals with the T₁, T₂, and diffusion contrasts (26-36). However, in practice, it is a challenge to label CSF without attenuating other issues and vice versa. Therefore, there remains a need for improved MRI systems and methods regarding water exchange processes in the brain.

### SUMMARY

An embodiment of the present invention is a system for magnetic resonance imaging of water exchange processes. The system includes a primary magnet configured to provide a magnetic field over an imaging volume, a magnetic gradient coil configured to generate a spatial encoding in the magnetic field, a radiofrequency (RF) coil, and a data processor. The RF coil is configured to acquire, from the imaging volume, at multiple time points, multiple water magnetic resonance signals, the water magnetic resonance signals including a first subset of signals that are labeled for a water exchange process and a second subset of signals that are not labeled. The data processor is configured to generate, from the first subset of signals, multiple labeled images, and generate, from the second subset of signals, multiple control images corresponding to the plurality of labeled images. The data processor is further configured to calculate one or more parameters to characterize a water exchange process in the imaging volume between a first water compartment and a second water compartment based on the labeled images and the corresponding control images.

Another embodiment of the present invention is a method for magnetic resonance imaging of water exchange processes. The method includes receiving multiple water magnetic resonance signals that were acquired by a radiofrequency (RF) coil from an imaging volume at multiple time points, the water magnetic resonance signals including a first subset of signals that were labeled for a water exchange process and a second subset of signals that were not labeled. The method further includes generating, from the first subset of signals, multiple labeled images, and generating, from the second subset of signals, multiple control images corresponding to the labeled images. The method further includes calculating one or more parameters to characterize a water exchange process in the imaging volume between a first water compartment and a second water compartment based on the labeled images and the corresponding control images.

### BRIEF DESCRIPTION OF THE DRAWINGS

Further objectives and advantages will become apparent from a consideration of the description, drawings, and examples.

FIG. 1A is a schematic illustration of a system for magnetic resonance imaging of CSF water exchange processes according to some embodiments.

FIG. 1B shows an illustration of the T₁-based phase alternate labeling with null recovery (T₁-PALAN) sequence.

FIG. 1C shows an illustration of the apparent diffusion coefficient-based phase alternate labeling with null recovery (ADC-PALAN) sequence.

FIG. 1D shows an illustration of the T₂-based phase alternate labeling with null recovery (T₂-PALAN) sequence.

FIG. 2A shows CSF signal as a function of inversion time (TI) for the third and lateral ventricles.

FIGS. 2B, 2C, 2D, 2E, and 2F show whole brain images for different TI values.

FIG. 3A shows averaged parenchyma recovery curves for the whole slice post control/label pulse (n=3) as a function of the post labeling time (PLD) for the T₁-based null recovery with phase alternate labeling (T₁-PALAN) sequence.

FIG. 3B shows the difference between control and label of the parenchyma recovery curves in FIG. 3A. The solid line is the theoretical fitting curve with Eq. 4 (R²=0.97).

FIG. 3C shows averaged CSF recovery curves for lateral ventricle (LV) and the third ventricle (3V) with the post control/label pulse (n=3) as a function of the PLD for the T₁-PALAN sequence.

FIG. 3D shows the difference of the averaged CSF recovery curves in FIG. 3C, i.e., the CSF water exchange kinetic curve, as a function of PLD. The solid line is theoretical fitting curves with Eq.7 (R²=0.99).

FIG. 3E shows the T₁-PALAN ΔS values for the rostral and caudal LV.

FIGS. 3F and 3I show high-resolution T₂ weighted images by Long TE-TSE for the two slices collected. Choroid plexuses (CP) are indicated with arrows.

FIGS. 3G and 3J show typical control images of the slices from FIGS. 3F and 3I for the T₁-PALAN embodiment.

FIGS. 3H and 3K show T₁-PALAN ΔS maps corresponding to FIGS. 3G and 3J.

The regions with hyperintensity ΔS values are indicated with arrows.

FIG. 4A shows averaged parenchyma recovery curves for the whole slice after the control/label pulse (n=3) as a function of the post labeling time (PLD) for the apparent diffusion coefficient-based null recovery with phase alternate labeling (ADC-PALAN) sequence.

FIG. 4B shows the difference of the parenchyma recovery curves in FIG. 4A. The solid line is the theoretical fitting curve in Eq.4 (R²=0.97).

FIG. 4C shows averaged CSF recovery curves with the control/label pulse (n=3) as a function of the PLD for the ADC-PALAN sequence.

FIG. 4D shows the difference of the averaged CSF recovery curves in FIG. 4C, i.e., ADC-PALAN kinetic curve, as a function of PLD. The solid line is theoretical fitting curve with Eq. 7 (R²=0.95).

FIG. 4E shows the ADC-PALAN ΔS values for the rostral and caudal lateral ventricle (LV).

FIG. 5A shows averaged CSF recovery curves for the CSF in ventricles after the control/label pulse (n=3) as a function of the post labeling time (PLD) for the T₂ based null recovery with phase alternate labeling (T₂-PALAN) sequence.

FIG. 5B shows the difference of the CSF recovery curves for the T₂-PALAN sequence in FIG. 5A. The solid line is the theoretical fitting curve with Eq.5 (R²=0.93). A label efficiency of 0.28 was obtained from the fitting.

FIG. 5C shows averaged parenchyma recovery curves with the control/label pulse (n=3) as a function of the PLD for the T₂-PALAN sequence in FIG. 5A.

FIG. 5D shows the difference of the averaged parenchyma recovery curves (n=3) in FIG. 5C, i.e., the CSF-ISF flow (CIF) kinetic curve, as a function of PLD. The solid line is the simulated curve using Eq. 7b by assuming ICF=20 ml/100 mg/min, ITT-0 ms, R_{1CSF}=0.33 s⁻¹ and R_{1/ISF}=0.56 s⁻¹.

FIGS. 5E and 5H show typical control images by the T₂-PALAN method for two slices.

FIGS. 5F and 5I show label images by the T₂-PALAN method for the two slices in FIGS. 5E and 5H.

FIGS. 5G and 5J show CIF ΔS maps, corresponding to FIGS. 5F and 5I, obtained by T₂-PALAN method following Eq. 7b for PLD=2s. The typical ROIs used for extracting CIF ΔS values are indicated in FIG. 5H.

FIG. 6 shows a schematic diagram of the CSF water exchange processes in ventricles that can be detected by the PALAN embodiments.

FIG. 7A shows the simulated T₁-PALAN ΔS signal as a function of PLD for different residue CSF signals.

FIGS. 7B, 7C, and 7D show the averaged T₁-PALAN ΔS signal of water phantom (n=3) as a function of PLD for different TI values.

FIGS. 8A, 8B, and 8C show the averaged T₁-PALAN ΔS signal of mouse ventricles (third and lateral ventricle) (n=3) as a function of PLD for different TI values.

FIG. 9 shows the simulated residual CSF signal with respect to the TI variation (ΔT₁) and T₁ difference (ΔT₁).

FIG. 10 shows the CSF and parenchyma MRI signals as a function of b values with the PGSE module.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed, without departing from the broad concepts of the current invention. All references cited anywhere in this specification, including the Background and Detailed Description sections, are incorporated by reference as if each had been individually incorporated.

Some embodiments of the invention provide a novel MRI strategy, referred to as Phase Alternate LAbeling with Null recovery (PALAN), that nulls one of the components (CSF or ISF) and labels the other components by flipping the phase of pulses, and may be used in some embodiments for quantifying (1) the interstitial to cerebrospinal fluid flow (ICF) and (2) cerebrospinal to interstitial fluid flow (CIF) in the brain. The water exchange between CSF and other tissues may shape the recovery curve of CSF or ISF. Here, other tissues include but are not limited to parenchyma, ependyma layer, blood, and choroid plexuses (CP). CSF water exchange measured via T₁, T₂, and ADC difference are referred to as T₁-PALAN, T₂-PALAN, and ADC-PALAN, respectively. Quantitative PALAN sequences provide novel tools to further understand bi-directional water exchanging processes in the brain between cerebrospinal fluid and the surrounding brain tissues.

In some embodiments of both T₁-PALAN and apparent diffusion coefficient (ADC)-PALAN MRI, the cerebrospinal fluid (CSF) signal is nulled by the T1 or diffusion module, while the partial recovery of other tissues (e.g., residual interstitial fluid) with short T₁ (T₁-PALAN) or low ADC values (ADC-PALAN) is labeled by alternating the phase of pulses. The water exchange is extracted from the difference between the recovery curves of CSF with and without labeling. Specifically, in some embodiments of T₁-PALAN and ADC-PALAN, the CSF is nulled while the ISF signal is maintained at Z-direction for control images. At the time that the CSF is nulled, ISF is inverted or saturated for label images by alternating the phase of pulses. After the T1 or ADC module, a post-labeling delay (PLD) is applied before the final MRI readout. A turbo spin echo (TSE) MRI with long echo time (LE-TSE) is implemented to read the CSF signal and to suppress the parenchyma signal. Then, the difference of the null recovery curves of CSF between label and control images can be used to extract ICF.

Similarly, CIF can be measured by labeling CSF due to its significantly different T2 value from ISF/parenchyma with T₂-PALAN. In some embodiments of T₂-PALAN, a Carr-Purcell-Meiboom-Gill (CPMG) T2 preparation module with long echo time is applied to null the parenchyma signal, while the CSF signal is maintained due to its extremely long T2. The first or last 90-degree pulse in the T2 module is alternated to flip the CSF magnetization up and down/saturation to create control and label images. Then, the difference of the null recovery curves of brain parenchyma signal between label and control images can be used to extract CIF.

Experiments were conducted using some embodiments of T₁-PALAN and ADC-PALAN to observe a rapid occurrence of CSF water exchange with the surrounding tissues at 67±56 ms and 13±2 ms transit times, respectively. The T₁ and ADC-PALAN signal peaked at 1.5 s. The CSF water exchange was 1153±270 ml/100 ml/min with T₁-PALAN in the third and lateral ventricles, higher than 891±60 ml/100 ml/min obtained by ADC-PALAN. T₁-PALAN ΔS values for the rostral and caudal ventricles are 0.15±0.13 and 0.034±0.01 (p=0.22, n=5), while similar ΔS values in both rostral and caudal lateral ventricles were observed by ADC-PALAN (3.9±1.9×10⁻³ vs 4.4±1.4×10⁻³: p=0.66 and n=5). The results from T₂-PALAN suggested the ISF exchanging from ependymal layer to the parenchyma was extremely slow: The results indicate that some PALAN embodiments are suitable tools to study CSF water exchange across different compartments in the brain. These experiments are described in further detail below:

### Methods

### MRI Experiments

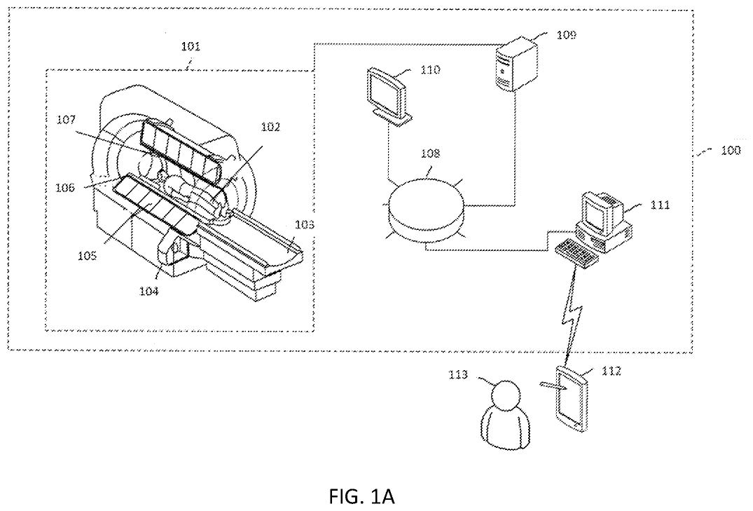

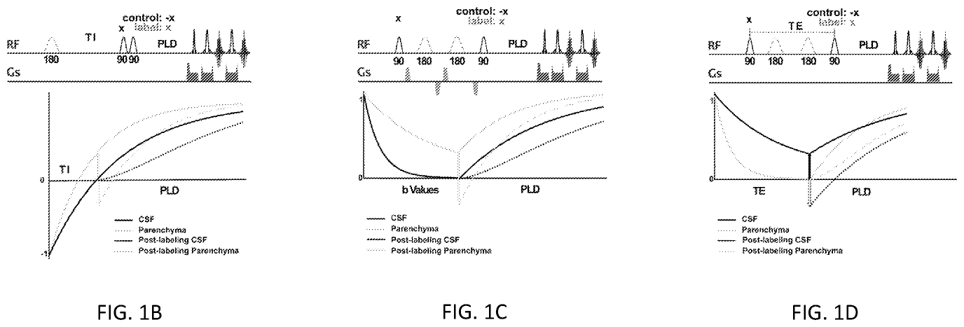

FIG. 1A shows a system 100 for magnetic resonance imaging of CSF water exchange processes according to some embodiments. The system 100 includes an MRI system 101. The MRI system 101 can accommodate a subject 102 under observation on scanner bed 103, for example. The MRI system 101 can include, but is not limited to, a primary magnet system 105 providing a substantially uniform (measured as parts-per million (ppm) within a certain diameter of spherical volume (DSV), e.g. <1 ppm over 40 cm for a clinical 3.0T scanner), main magnetic field B₀ (e.g., 1.5T or 3.0T) for a sample 102 (subject or object) under observation on scanner bed 103, a magnetic gradient coil system 106 providing a perturbation of the main magnetic field B₀ to encode spatial information of the constituent molecules of subject 102 under observation, and a radiofrequency (RF) coil system 107 to transmit electromagnetic waves and to receive magnetic resonance signals from subject 102. The RF coil system 107 may include separate radiofrequency transmit and receive coils, each having a plurality of coils. For instance, receivers can have multiple MRI detectors, such as those provided in an MRI phased-array.” Some embodiments of the invention include 16, 32, 60, or 120 MRI detectors, though these numbers are provided as examples, and the embodiments of the invention are not limited to these examples. Each MRI detector has a spatial sensitivity map.

The system 100 also has a processor 109 configured to communicate with the MRI system 101. The processor 109 can be partially or totally incorporated within a structure 104 that houses the MRI system 101 and/or partially or totally incorporated in a computer, a server, or a workstation that is structurally separate from and in communication with the MRI system 101.

The system 100 can include a data storage unit 108 that can be, for example, a hard disk drive, a network area storage (NAS) device, a redundant array of independent disks (RAID), a flash drive, an optical disk, a magnetic tape, a magneto-optical disk, or that provided by local or remote computer ‘cloud networking, etc. However, the data storage unit 108 is not limited to these particular examples. It can include other existing or future developed data storage devices without departing from the scope of the current invention.

The processor 109 can be configured to communicate with the data storage unit 108. The processor 109 can also be in communication with a display system 110 and/or a console station 111. In some embodiments, results can be displayed by the display system 110 or the console station 111. In some embodiments, an operator 113 may use an input/output device 112 to interact, control, and/or receive results from system 100.

The MRI system 101 is configured to apply a plurality of spatially localized MRI sequences, wherein each sequence is adjusted to be sensitive to an MRI parameter whose measurement requires the acquisition of a plurality of spatially localized MR signals. The MRI system 101 is configured to adjust at least one of the applied plurality of spatially localized MRI sequences so as to substantially fully sample an image k-space of the sample, and adjust the remainder of the applied plurality of spatially localized MRI sequences to under-sample the image k-space of the sample. The MRI system 101 is configured to acquire a plurality of image k-space MRI signal data sets, each responsive to the application of each of the plurality of spatially localized MRI sequences. The processor 109 is configured to estimate a sensitivity map of each of the plurality of MRI detectors using a strategy to suppress unfolding artefacts, wherein the strategy is based on data acquired from the substantially fully-sampled spatially localized MRI sequence. The processor 109 is configured to apply the estimated sensitivity maps to at least one of the image k-space MRI signal data sets to reconstruct a spatial image of MRI signals that are sensitive to the MRI parameter within a support region of the spatial image in which the sample resides.

According to some embodiments of the invention, the MRI system 101 and the processor 109 are associated by one of an Ethernet connection, a Wi-Fi connection, or by integration of the processor 109 into the MRI system 101.

According to some embodiments, the processor 109 is configured to reconstruct an image whose intensity is directly proportional to a spatial distribution of the MRI parameter within the sample 102, and the display system 110 or the console station 111 is configured to display the reconstructed image.

In this study, the MRI system 101 was a horizontal bore 11.7 T Bruker Biospec system (Bruker, Ettlingen, Germany). For these experiments, the RF coil system 107 included a 72 mm quadrature volume resonator and a 2×2 mouse phased array coil as transmitter and receiver.

Ten female mice (C57BL/6J) aged 11-12 months were used for this study. All animals were anesthetized using 2% isoflurane in medical air, followed by 1% to 1.5% isoflurane for maintenance during the MRI scan. The respiratory rate was monitored via a pressure sensor (SAII, Stony Brook, NY, USA) and maintained at 70-90 breaths per minute. The B₀ field over the image slice was adjusted using field mapping and second-order shimming.

### MRI Pulse Sequences

FIG. 1B shows an illustration of the T₁-based phase alternate labeling with null recovery (T₁-PALAN) sequence of some embodiments, acquired with the system of FIG. 1A and used for these experiments. The signal changes of CSF and tissue without labeling are solid lines: after labeling are dashed lines. After the inverting pulse, a pair of 90-degree pulses, i.e., Z-filter, is applied at the CSF null time. The phase of the second 90-degree pulse in the Z-filter is alternated and can flip the tissue longitudinal magnetization up (control) and down (label). The difference between the dashed and the solid CSF recovery curves is due to the water exchange between CSF and the surrounding tissues. A TSE MRI with a long echo time (Long TE-TSE) is implemented to readout the CSF signal and suppress the tissue signal.

FIG. 1C shows an illustration of the apparent diffusion coefficient-based phase alternate labeling with null recovery (ADC-PALAN) sequence of some embodiments, acquired with the system of FIG. 1A and used for these experiments. The signal changes of CSF and tissue without labeling are solid lines: after labeling are dashed lines. A twice-refocused pulsed gradient spin echo (PGSE) module is applied to suppress the CSF signal with high b values, while a strong tissue signal is preserved. The phase of the second 90-degree pulse in the PGSE is alternated to flip the tissue longitudinal magnetization up (control) and down (label). Similar to T₁-PALAN, the water exchanging process introduces the difference in CSF recovery curves and Long TE-TSE is used for the CSF imaging.

FIG. 1D shows an illustration of the T₂-based phase alternate labeling with null recovery (T₂-PALAN) sequence of some embodiments, acquired with the system of FIG. 1A and used for these experiments. The signal changes of CSF and parenchyma without labeling are solid lines, after labeling are dashed lines. In this embodiment, a Carr-Purcell-Meiboom-Gill (CPMG) module is applied to null the ISF/parenchyma signal. The recovery curve of ISF is modulated by the flow from CSF to ISF.

For the T₁-PALAN embodiment, a hyperbolic secant (HS) inversion pulse (15 ms) was applied. At the CSF null inversion time (TI_{null,CSF}=2 s), the CSF signal was at the zero baselines while part of the ISF signal had recovered. One Z-filter composited by two 90-degree pulses (Gaussian pulse with 1.4 ms width) was applied at the TI_{null,CSF}. The phase of the second 90-degree pulse in the Z-filter was alternated by 180-degree, flipping the longitudinal tissue magnetization up (control) and down (label). CSF recovery during eleven post-labeling delays (PLDs) (0, 0.1, 0.2, 0.4, 0.6, 1, 1.5, 2, 3, 4, 5 s) were recorded. The water exchange between CSF and other tissues was the difference between the CSF recovery curves with and without labeling. The recovery from the labeling pulse was different from the original recovery curve due to CSF water exchange. Theoretically, the label images in the T₁-PALAN can also be recorded with a 180-degree pulse. However, the water magnetization can be attenuated during the labeling pulses due to the rotating frame relaxation, which will slightly alternate the null time. Unwanted artifacts can be introduced during the process. A Z-filter can solve this issue by using two identical pulses for both control and label images. Four pairs of control/label parenchyma signals were collected to measure the labeling efficiency, and eight pairs of CSF signals were recorded for the CSF water exchange measurement. A turbo spin-echo (TSE) MRI with long echo time (Long TE-TSE) was implemented to read the CSF signal and to suppress the parenchyma signal with TE=245 ms, pre-scan delay 5 s, TSE factor=96, slice thickness =1 mm, a matrix size of 96×96, and a resolution of 0.17×0.17 mm².

The T₁ relaxation time of the mouse brain CSF was measured with the inversion recovery TSE (TE=222 ms) readout. TI=0.01, 1.2, 2.4, 3.6, 4.8, 6.0, 7.2, 8.4, 9.6, 10.8 s were used for the CSF with slice thickness 1 mm, matrix size of 96×96, and a resolution of 0.18×0.18 mm².

The ADC-PALAN embodiment, much like the T₁-PALAN embodiment, was also capable of measuring CSF water exchange. Instead of applying one T₁ preparation module, a twice-refocused pulsed gradient spin echo (PGSE) module (37) was applied to suppress the CSF signal with high b values (2100 s/mm²) and leave the recovered parenchyma signal untouched. The total PGSE module was 30 ms, the gradient length was 3 ms. Mao pulses (38) with 6 ms width were used for the 180-degree pules, and Gaussian pulses (1.4 ms width) were used for the two 90-degree pulses in the PGSE module. The phase of the second 90-degree pulse in the PGSE module was alternated by 180-degree to obtain the control and label images.

In the T₁-PALAN and ADC-PALAN multi-PLD studies, a single axis slice was collected at −0.7 mm from the anterior commissure (AC) to cover the left ventricles (LV) and third ventricle (3V). Common belief states that CP is found in the caudal lateral ventricles (39,40). To further examine the possible contributions to the CSF water exchange in T₁-PALAN and ADC-PALAN embodiments, two high-resolution single PLD (1.5 s) slices (−0.7 and 0.9 mm from AC) were collected. T₁- and ADC-PALAN maps that covered the rostral and caudal LV (n=5) were obtained. High-resolution T₂ weighted images with a long TE turbo spin-echo (Long TE-TSE) sequence determined the CP locations. The scan time for each Long TE-TSE experiment was 13 minutes with TR/TE=6 s/106 ms, RARE factor=32, slice thickness=0.5 mm, a matrix size of 256×256 within a FOV of 16×16 mm².

The measurement of CSF backflow from ventricles to parenchyma, i.e., CIF, was achieved with the T₂-PALAN embodiment. A Carr-Purcell-Meiboom-Gill (CPMG) module was used in the T₂-PALAN to null the parenchyma ISF signal. The CIF process modulated the recovery curve of parenchyma. The label and control images were collected by alternating the phase of the second 90-degree pulse at the end of the CPMG module by 180-degree. TSE with short TE (TE=5 ms) was used to acquire parenchyma MRI images with a pre-scan delay of 5 s, TSE factor=16, slice thickness=1.5 mm, FOV=1.6×1.6 mm². A matrix size of 32×32 was used for the CIF kinetic curve measurement with four pairs of control/label images, and a matrix size of 64×64 was used for the high-resolution CIF images with 32 pairs of control/label images. The total experimental time for the high-resolution CIF images was 31 minutes. 9 PLDs (0, 0.5, 1, 1.5, 2, 2.5, 3, 4, and 6 s) were acquired for the CSF labeling efficiency measurement and the CIF buildup curves with the T₂-PALAN method. In the T₂-PALAN multi-PLD studies, a single axis slice was collected at −0.7 mm from AC for the CSF optimization, and the slice at −4.1 mm from AC was used for the parenchyma signal optimization. Two slices (−0.7 and −4.1 mm from AC) were collected for the high-resolution single PLD (1.5s) CIF maps.

### Data Analysis

All MRI images were processed using custom-written MATLAB scripts (MathWorks, R2020a). The CSF water exchange signals (ΔS_{CSF}) or tissue labeling signals (ΔSₜᵢₛₛᵤₑ) were calculated by subtracting the label and control images following:

$$Δ {S}_{CSF / tissue} = ( {S}_{control} - {S}_{label} ) / {S}_{0} . \qquad ( 1 )$$

The S₀ images were collected by setting PLD=10 s for tissue and 15 s for CSF in the PALAN embodiments.

The recovery of the tissue signal in the T₁-PALAN and ADC-PALAN after the T₁ or PGSE preparation module can be described as:

$${S}_{tissue}^{control} = {S}_{0} \vert 1 - ( 1 - α ) {e}^{- PLD \cdot {R}_{1 tissue}} \vert ( phase : - x ) , \qquad ( 2 )$$

$${S}_{tissue}^{label} = {S}_{0} \vert 1 - ( 1 + α ) {e}^{- PLD \cdot {R}_{1 tissue}} \vert ( phase : + x ) , \qquad ( 3 )$$

where α was the tissue signal after the Z-filter, i.e., the labeling efficiency. R₁ₜᵢₛₛᵤₑ was the T₁ relaxation rate of the tissue. In practice, nonzero noise background was present in the MRI image due to the magnitude Rician noise. Therefore, the reliable way of extracting the labeling efficiency was fitting the difference image (sₜᵢₛₛᵤₑ^{control}−sₜᵢₛₛᵤₑ^{label})/S₀ following:

$$Δ {S}_{tissue} = [ \vert 1 - ( 1 - α ) {e}^{- PLD \cdot {R}_{1 tissue}} \vert - \vert 1 - ( 1 + α ) {e}^{- PLD \cdot {R}_{1 tissue}} \vert ] . \qquad ( 4 a )$$

Similarly, the difference between the two CSF recovery curves after the CPMG module in the T₂-PALAN method was given by:

$$Δ {S}_{CSF} = ( {S}_{CSF}^{control} - {S}_{CSF}^{label} ) / {S}_{0} = [ \vert 1 - ( 1 - α ) {e}^{- PLD \cdot {R}_{1 CSF}} \vert - \vert 1 - ( 1 + α ) {e}^{- P LD \cdot {R}_{1 CSF}} \vert ] , \qquad ( 4 b )$$

where R_{1CSF} was the CSF T₁ relaxation rate.

A standard single-compartment kinetic model was applied to quantify the water exchange from the observed CSF signal reduction ΔS_{CSF}, assuming instantaneous exchange of labeled spins from tissue or blood to ventricles. The observed CSF signal reduction was described by the difference between the sum over the series of delivered magnetization units to CSF from tissue or blood (the arterial input function, AIF) and the clearance of the magnetization by the relaxation of the CSF (the impulse residue function, IRF) (41-43).

The IRF function was given by:

$$c ( t ) = \{ \begin{matrix}0 ( t < WTT ) \\ 2 α \cdot f \cdot {e}^{- t {R}_{1 a}} ( t > WTT )\end{matrix} , \qquad ( 5 )$$

where α was the tissue labeling efficiency of the T₁ or PGSE preparation module, f was the water exchange in units of volume of tissue water delivered per volume of CSF per unit time (ml/100 ml/min). WTT was the water exchange transit time, i.e., the time for the tissue water to reach CSF after labeling. The R₁ₐ was a labeling decay rate due to the limited bolus duration.

The observed CSF reduction ΔS was the convolution of the AIF and the IRF functions, i.e.,

$$Δ {S}_{CSF} = c ⊗ {e}^{- t {R}_{1 CSF}} . \qquad ( 6 )$$

$$Then ,$$

$$Δ {S}_{CSF} = \{ \begin{matrix}0 ( PLD < WTT ) \\ \frac{2 α \cdot f}{{R}_{1 app}} {e}^{- PLD \cdot {R}_{1 α}} ( {e}^{( PLD - ITT ) \cdot {R}_{1 app}} - 1 ) ( PLD > WTT )\end{matrix} , \qquad ( 7 a )$$

where R₁ₐₚₚ=R₁ₐ−R_{1CSF}. When measuring CIF rate by ΔS_{Parenchyma}, the same equation can be used except the R₁ₐₚₚ=R_{1CSF}−R_{1/SF} and:

$$Δ {S}_{tissue} = \{ \begin{matrix}0 ( PLD < ITT ) \\ \frac{2 α \cdot f}{{R}_{1 app}} {e}^{- PLD \cdot {R}_{1 CSF}} ( {e}^{( PLD - ITT ) \cdot {R}_{1 app}} - 1 ) ( PLD > ITT )\end{matrix} , \qquad ( 7 b )$$

where f was the CIF flow in units of volume of CSF delivered per gram ISF per unit time (ml/100 mg/min).

A paired-sample t-test was performed to compare between PALAN values of the rostral and caudal LV using the MATLAB built-in function “ttest”. A t-test is considered statistically significant for p<0.5 and highly significant when p<0.001.

### Results

### In Vivo Inversion Time Optimization

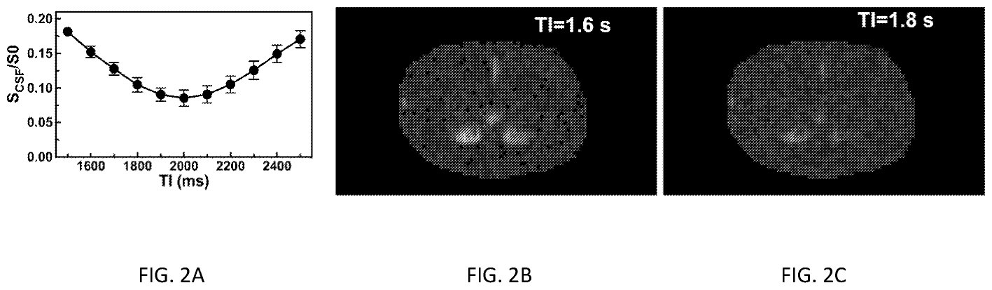

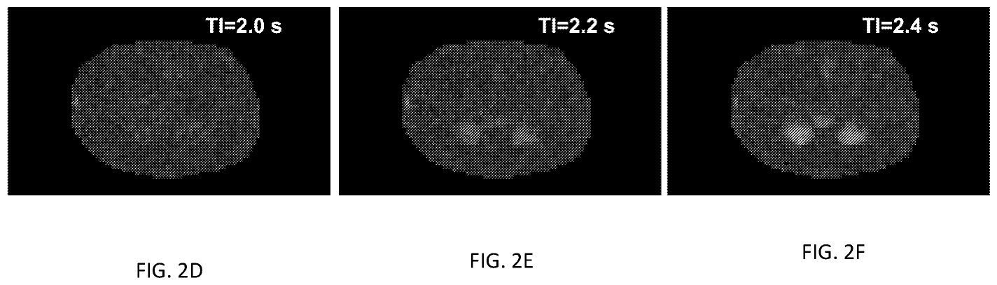

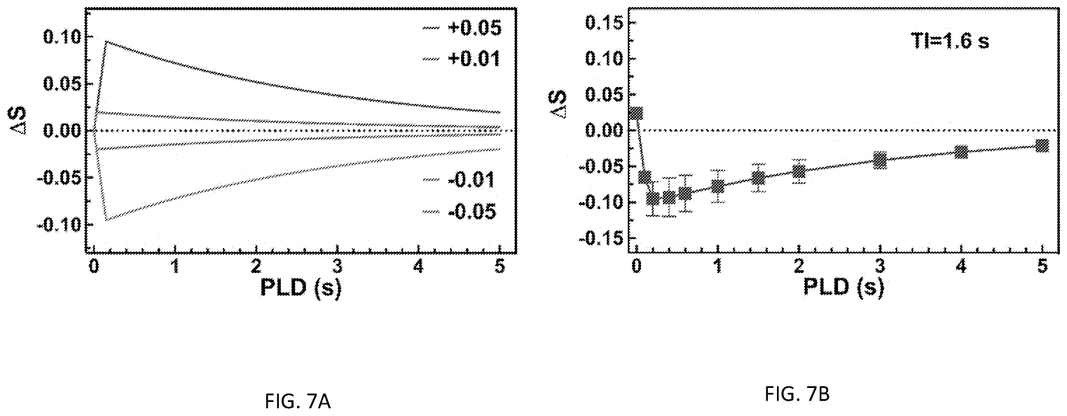

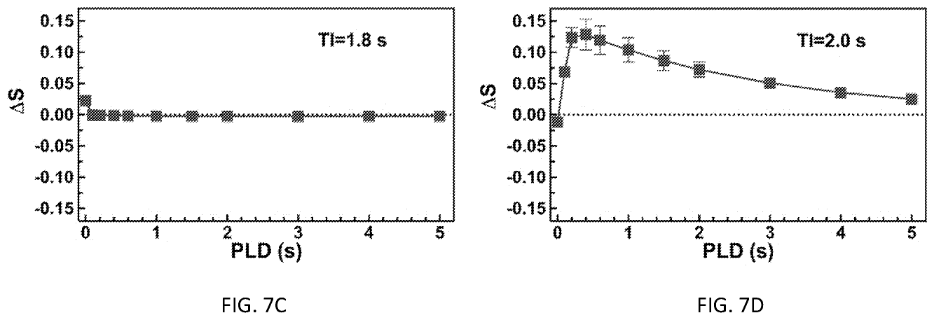

The successful application of T₁-PALAN for water exchange measurement relied on the complete suppression of CSF signal when partially recovered tissue signals are labeled, which is determined by inversion time (TI). Validation using the water phantom (See Supplemental Materials section below) showed that the control and label image canceled each other with a residual water signal less than 0.003 (see FIGS. 7A-7D, described below). The CSF signal intensity as a function of T₁ was measured before performing in vivo PALAN experiments, and the results are plotted in FIGS. 2A-2F together with the typical whole-brain images with different TI values. FIG. 2A shows the CSF signal as a function of T₁ for the third and lateral ventricles. The typical whole brain images for different TI values are 1.6 (FIG. 2B), 1.8 (FIG. 2C), 2.0 (FIG. 2D), 2.2 (FIG. 2E) and 2.4 s (FIG. 2F). At TI=2 s, the CSF signal is completely suppressed, as demonstrated in FIGS. 2A and 2D. Due to the magnitude of Rician noise, the CSF signal is not zero with TI=2 s in FIG. 2A.

### ISF to CSF Water Exchange Measured by T₁-PALAN

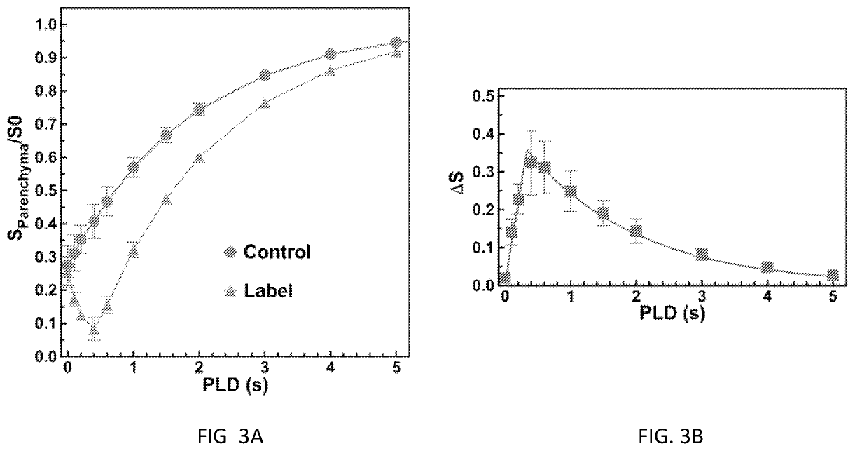

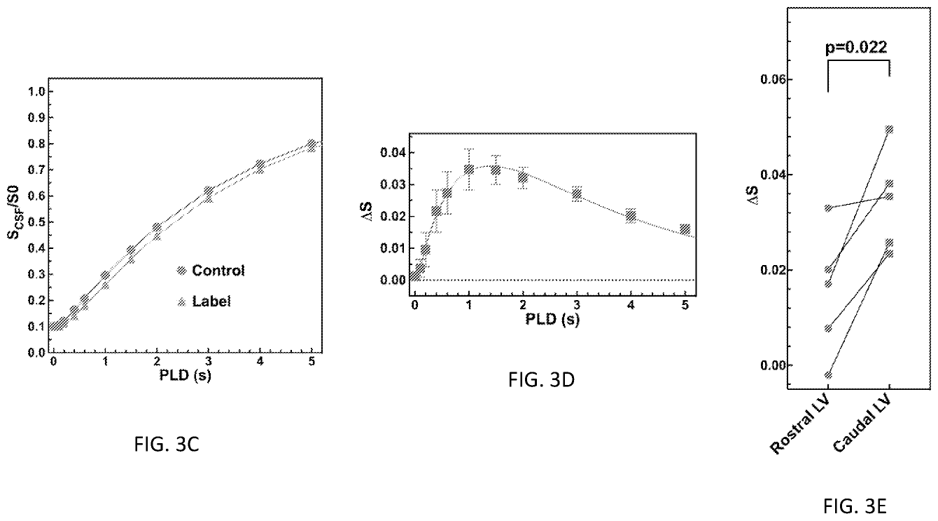

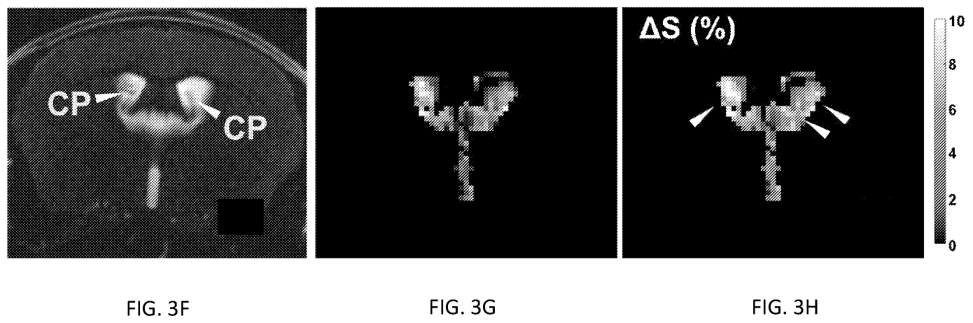

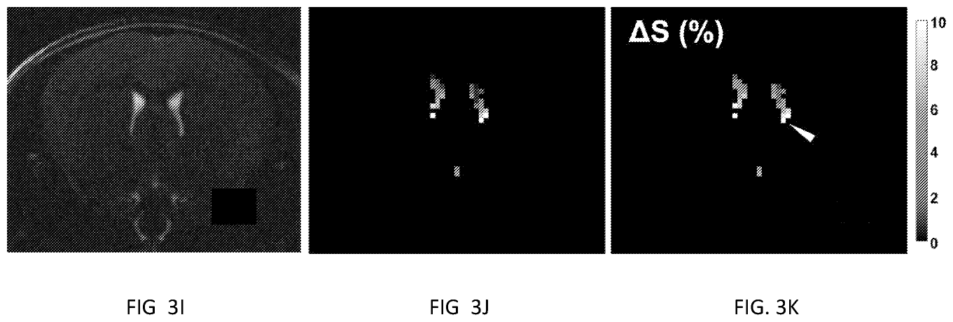

The current study used a large volume coil that covered the entire mouse brain. Therefore, the water diffusion and flow outside the imaging slices did not alternate the T₁-PALAN ΔS signal since all preparation pulses were non-selective pules. The result was further supported by the water phantom study (see FIG. 7C, described below). The typical T₁-PALAN CSF and parenchyma recovery curves, together with the ΔS maps, are shown in FIGS. 3A-3D. The post-labeling parenchyma signal curve shows a typical inversion recovery curve. Theoretically, the MRI signals with PLD less than 0.4 s were negative after the labeling pulse as shown in the standard inversion recovery curves, but the usage of magnitude-reconstructed images resulted in positive values. The parenchyma signal difference curves using Eq. 4a yielded the labeling efficiency of α=0.21±0.2. The CSF recovery curves show a clear difference between control and label pulses, indicating high water exchange values. The magnitude Rician noise background led to a positive CSF signal at PLD=0 s. The CSF difference curves, i.e., T₁-PALAN kinetic curves, show a clear buildup and decay pattern that peaks at PLD=1.5 s. The curves were fitted to Eq. 7a, which gave CSF water exchange value 1153±270 ml/100 ml/min, and WTT=67±56 ms. These values are summarized in Table 1, which shows averaged CSF water exchange and water exchange transit time (WTT) values obtained with both the T₁-PALAN and ADC-PALAN embodiments (n=3).

TABLE 1

| | CSF water exchange | WTT |
|---|---|---|
| | ml/100 ml/min | ms |
| T₁-PALAN | 1153 ± 270 | 67 ± 56 |
| ADC-PALAN | 891 ± 60 | 13 ± 2 |

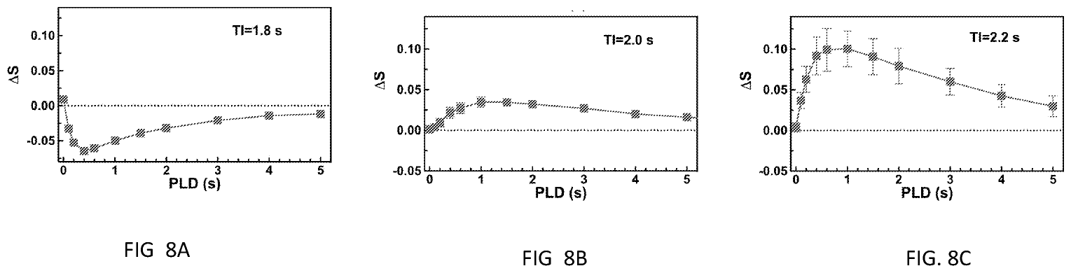

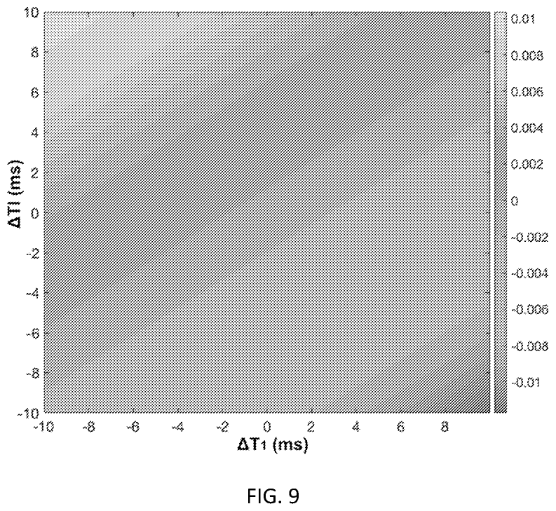

R_{1CSF}=0.33 s⁻¹ was determined by the inversion recovery sequence and was used in the exchange rate calculation for both T₁- and ADC-PALAN. In the calculation, all tissue labeling efficiencies were assumed to be identical to the parenchyma. The T₁-PALAN ΔS values for the rostral and caudal LV are 0.15±0.13 and 0.034±0.01 (p=0.22, n=5), respectively (FIG. 3E). The residual CSF signal with TI=2 s can be estimated from the signal at PLD=0.1 s, which was less than 0.0037 and was neglectable compared to the peak ΔS signal (see FIGS. 8A-8C, described below). The impact of imperfect T₁ and T₁ on the residual CSF signal was simulated and plotted in FIG. 9, which is described below. The residual signal is less than 0.006 with a T₁ error of 10 ms, disregarding T₁ variation.

FIGS. 3E-3J are typical T₁-PALAN control images and ΔS CSF maps of the brain ventricles. The high-resolution T₂ maps show the CP in the LV (FIGS. 3F and 3I), and the CP is visible in the caudal LV regions. The CSF water exchange values were calculated from Eq. 7a with R₁ₐ=1.4 s⁻¹, WTT=67 ms, and a labeling efficiency of 0.21 for all tissues. Across the ventricles, the water exchange rates were not uniform, and most water flowed from the bottoms of the LV, as indicated in FIGS. 3H and 3K with arrows.

### ISF to CSF Water Exchange Measured by ADC-PALAN

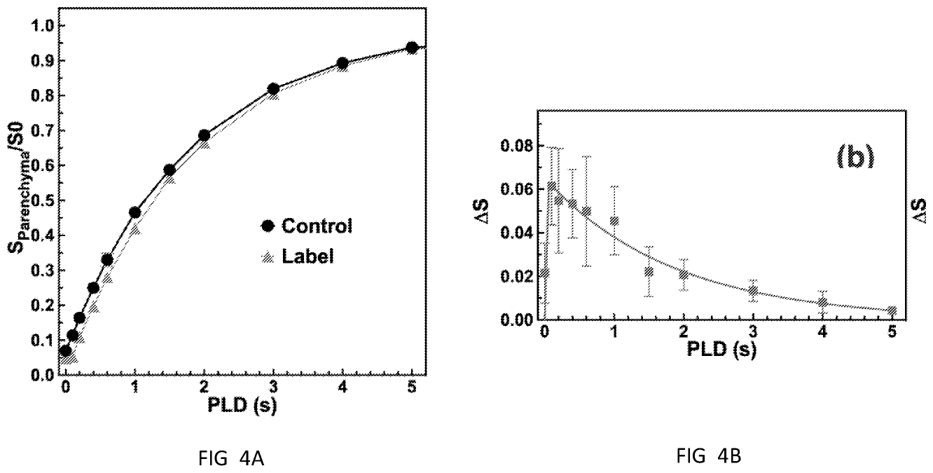

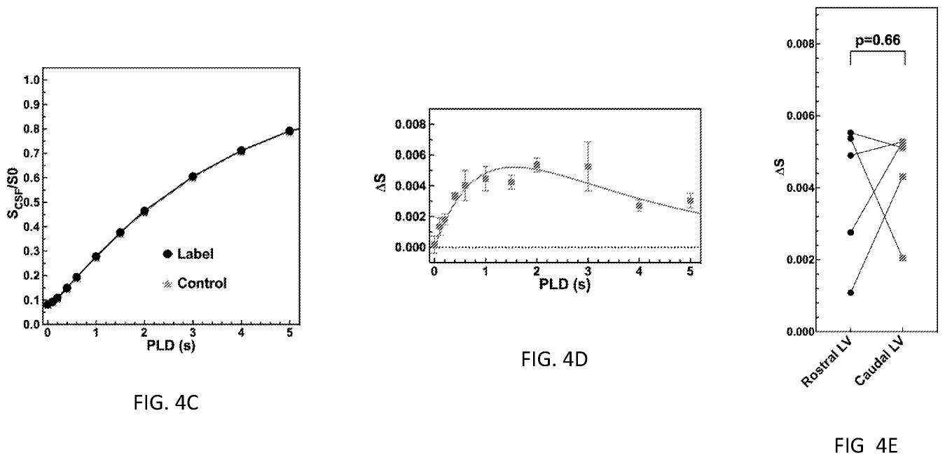

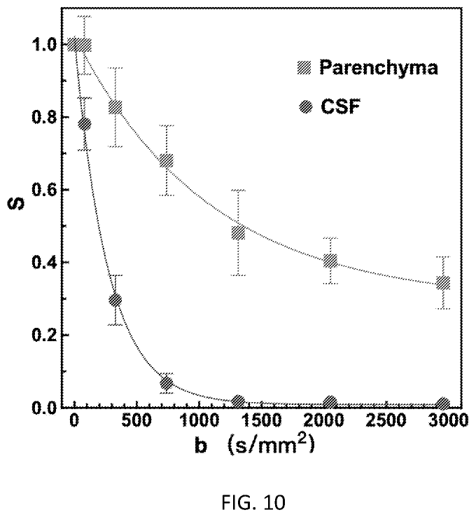

CSF and parenchyma (tissue) optimization were performed as a function of the PLD for ADC-PALAN shown in FIGS. 4A-4E. As suggested by the b-dependent ADC measurement on the parenchyma and CSF (see FIG. 10 described below), the CSF signal was less than 1% with b=2100 s/mm² than the signal with b −0 s/mm². The difference between label and control signals yielded a labeling efficiency of 0.3±0.12. Due to the signal reduction introduced by ADC and T₂, the observed labeling efficiency for parenchyma was significantly lower than those of the T₁-PALAN embodiment, which led to a much-reduced ADC-PALAN ΔS signal of 4.2±0.5×10³ (PLD=1.5 s) compared to 35±4.5×10³ (PLD=1.5 s) in the T₁-PALAN embodiment. Eq. 7a fitted perfectly with the ADC-PALAN kinetic curve and gave CSF water exchange value 891±60 ml/100 ml/min, and WTT=13±2 ms (Table 1). The ADC-PALAN ΔS values for the rostral and caudal LV were 3.9±1.9×10⁻³ and 4.4±1.4×10³ (p=0.66, n=5), respectively. The much lower signal obtained by the ADC-PALAN embodiment was a barrier to obtaining the ADC-PALAN ΔS maps.

### CSF to ISF Water Exchange Measured by T₂-PALAN MRI

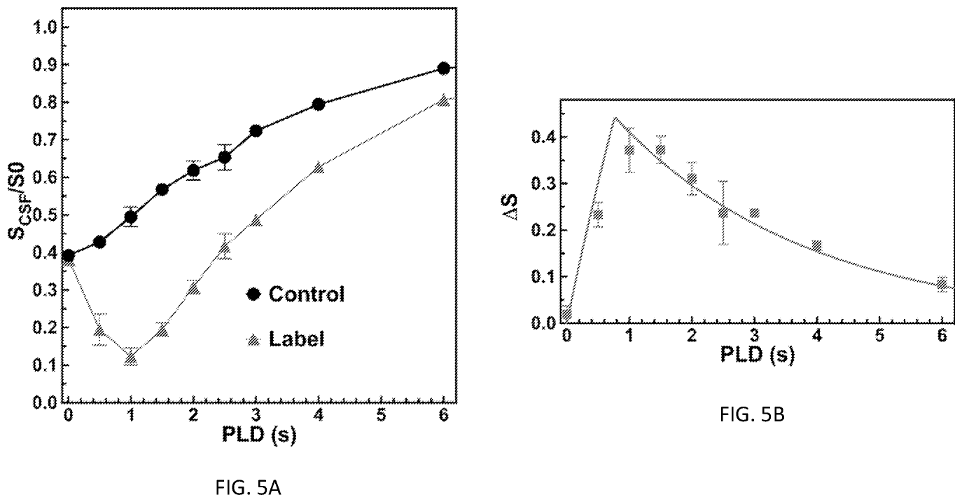

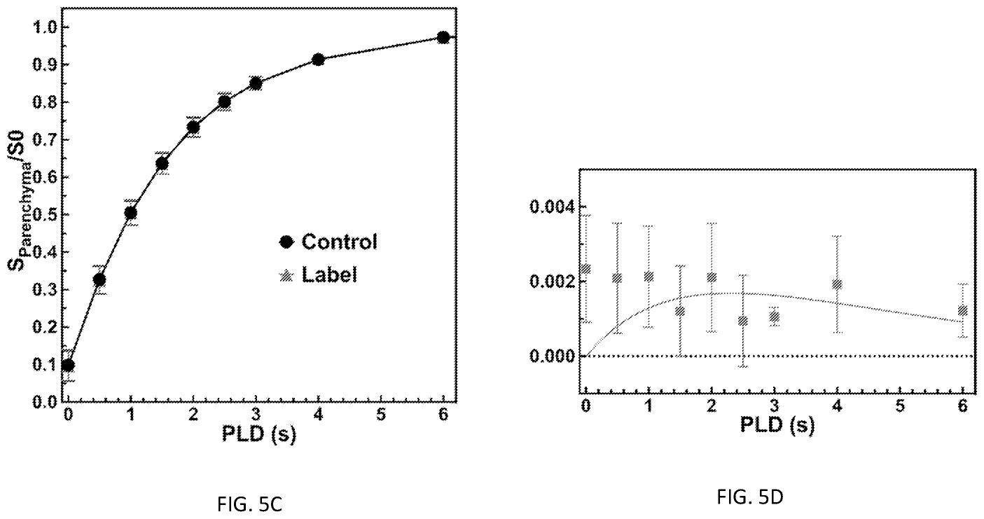

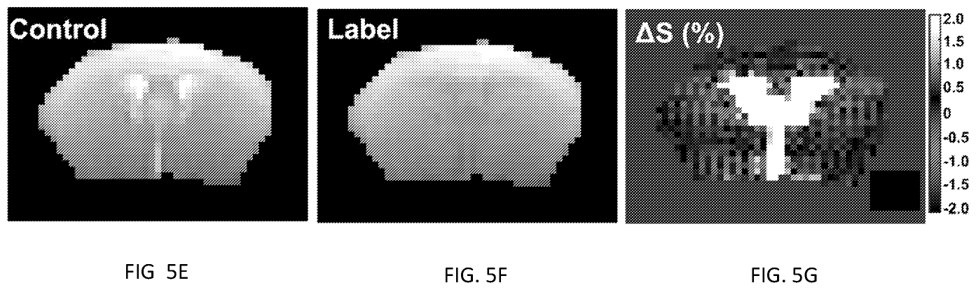

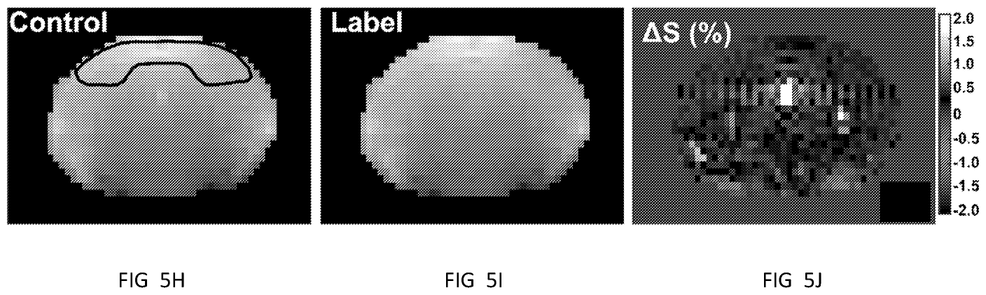

The backflow from CSF to parenchyma i.e., CIF was measured with the T₂-PALAN embodiment, and the results are presented in FIGS. 5A-5J. Differently from the ADC and T₁-PALAN embodiment, the CSF signal was maintained and labeled in T₂-PALAN. The CSF recovery difference between the post-labeling and the original curves with Eq. 4b gives the labeling efficiency of 0.28±0.3. The parenchyma recovery curves after the label/control pulse (FIG. 5C) shows a barely noticeable difference, which indicates a negligible flow rate from CSF to the parenchyma. The CIF ΔS signal is under 0.22%. (FIG. 5D) To obtain a reliable estimate of the ICF values, we addressed the labeled CSF strong interference and the overall low signal in the map (FIG. 5G) by performing the high-resolution CIF maps with 32 averages at a slice with much fewer ventricles, i.e., −4.1 mm from AC (FIG. 5H-5J). The overall CIF ΔS signals for the ROIs shown in FIG. 5H were found to be 0.076±0.027% at PLD=2 s (n=3) since simulation found the maximum ICF signal at 2s. The maximum CIF value was estimated to be 15±6 ml/100 mg/min with Eq. 7b, assuming ITT=0 ms, R_{1CSF}=0.33 s⁻¹ and R_{1/ISF}=0.56 s⁻¹.

### DISCUSSION

As described above, a series of non-invasive PALAN MRI embodiments were to measure the CSF water exchange with its surrounding tissues by selectively labeling the water in the tissues with T₁ and ADC contrasts. The T₁-PALAN pulse sequence embodiment provided much higher signals than the ADC-PALAN embodiment. Both embodiments of T₁-PALAN and the ADC-PALAN used herein suggested that the water exchange between CSF and other tissues occurs rapidly. On the contrary, CSF to the parenchymal regions far away from ventricles was a pronouncedly slow process and barely observable with the spin labeling method.

T₁-PALAN has its advantage in terms of obtaining the kinetic curves. In this study, T₁ₐ=1/R₁ₐ=0.7 s was found, far less than the parenchyma T₁ relaxation time of 1.8 s. Therefore, the bolus duration generated by the T₁ module was on the order of 0.7 s. As previous studies suggested, the blood signal can be suppressed as low as 400 s/mm² (44,45). Therefore, the blood signal was fully suppressed by the high b value (2100 s/mm²) in the ADC-PALAN and could not be labeled.

In contrast, any tissues with shorter TI values than CSF can be labeled by T₁-PALAN. The effect T₁ of the blood signal is very short due to the blood in-flow effect and the blood labeling efficiency approached one. Therefore, the T₁-PALAN values theoretically contained strong signals from the blood through CP, i.e., blood-cerebrospinal fluid barrier arterial spin labeling (46). The higher CSF water exchange value (1153±270 ml/100 ml/min) by T₁-PALAN than the value measured by the ADC-PALAN (891±60 ml/100 ml/min) confirms that T₁-PALAN contains signals from the blood. The contamination from CP water exchange in T₁-PALAN was further validated by the significantly different T₁-PALAN ΔS values of the rostral and caudal LV (FIG. 3E). The result indicated that the water exchange between CP and CSF could be as high as 50% in the caudal LV. This water contribution includes the blood flow from CP to the ventricle and the exchange between CSF and CP tissue. The similar CSF water exchange values measured by ADC-PALAN in rostral and caudal LV suggested that the observed CSF water exchange signal mainly came from parenchyma and ependyma, not blood. (FIG. 4E).

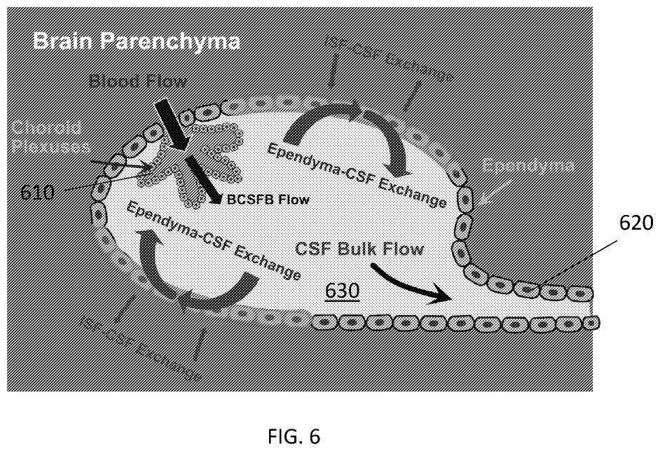

The possible contributions to the CSF water exchange are illustrated in FIG. 6. CSF is primarily secreted from the choroid plexuses (CP) 610 provided by the anterior choroidal arterial blood. There is a rapid water exchange between the intercellular water in the ependymal layer 620 and the CSF in the ventricles 630. However, the exchanging process from the ependymal layer 620 to the regions far away from ependyma, i.e., CSF to interstitial fluid (ISF) exchange, is an extremely slow process. Eventually, CSF flows out of ventricles 630 and exits into the subarachnoid space (SAS) at the cisterna magna (not shown).

The parenchyma, ependyma, CP can contribute to the measured T₁- and ADC-PALAN ΔS signal, while the blood from CP only contributes to the T₁-PALAN ΔS signal. The short bolus duration measured by both T₁- and ADC-PALAN embodiments suggested that the major water exchange occurs in the surface of the ventricles, i.e., the ependyma layers. The driving force that facilitates the rapid CSF-tissue water exchanging process is unknown. One possible explanation is the pulsatile nature of the CSF flux, which appears to be craniocaudally oriented during cardiac systole and in the reverse direction during diastole (47-49).

A recent pseudo-continuous ASL method has measured the blood-cerebrospinal fluid barrier (BCSFB) flow in mice to be 13-20 ml/100 ml/min, i.e., a total flow of 0.52-0.8 μL/min by assuming LV volume 4 μL (46). A Gd MRI agent cannot measure the BCSFB flow because of its inability to pass the BCSFB. However, D-glucose penetrates BCSFB with the help of abundant glucose transporter on the BCSFB. Hence dynamic glucose enhancement can be used to assess the BCSFB flow (23,24). Interestingly, the BCSFB ASL signal in mouse LV was mainly found at the top of both LV and no signal was observed in 3V. The T₁-PALAN hyperintensity regions are visible in 3V and at the bottom of the LV. (FIGS. 3H and 3K)

The non-invasive PALAN embodiments provided effective tools to reveal the CSF water exchange in the brain. The current study quantified the CSF water exchange, i.e., 891-1153 ml/100 ml/min in LV and 3V. A recent invasive study on mouse brain states the CSF outflow from both LV and 3V is on the order of magnitude of 0.1 μL/min (50), which is negligible compared to the CSF water exchange obtained by the current study (32-44 μL/min in 4 μL LV). The current result indicated that the CSF rapidly exchanges with the surrounding tissues, mainly with the ependymal layer, as indicated in FIG. 6. No fresh CSF is generated in this process. The difference between CSF secreted from CP (0.52-0.8 μL/min) (46) and the total outflow (0.1 L/min) (50) suggests that the surrounding parenchyma absorb a large portion of the CSF in ventricles. But due to the large mass of the parenchyma, kinetically speaking, the absorption is prolonged, e.g., less than 15 ml/100 mg/min according to measurements using T₂-PALAN. The current model explains the extremely high measured turnover rate of the CSF and perfectly explains the study of the radioactive-labeled substances in CSF, in which the infused tracer crosses the periventricular ependymal layer and can be found in the extracellular space (7,51,52).

In some embodiments, PALAN can measure the backflow from CSF to parenchyma by nulling parenchyma with a long T₂ preparation module, which uses the significant T₂ different between CSF and parenchyma. However, the extremely slow exchange between CSF and parenchyma making it a challenge to reliably extract the exchange rate as demonstrated in FIGS. 5A-5J. The low exchange rate between CSF and parenchyma suggests that the CSF water exchange mainly occurs between CSF and the ependyma layers on both CP and ventricles.

In summary, the PALAN MRI embodiments discussed above provide technically achievable tools to examine the association between CSF water exchange and functional decline in many neurodegeneration diseases. These embodiments complement the invasive MRI method with agents and complete view of the CSF water exchange in the ventricles with the surrounding tissues.

### CONCLUSION

T₁ and ADC PALAN schemes were presented as new translational MRI methods to quantify in vivo water exchange between CSF and the surrounding tissue in the mouse brain. PALAN offered CSF water exchange images with high sensitivity and quality. The CSF water exchange rate from the tissue to the ventricles was quantified with the T₁-PALAN and the ADC-PALAN embodiments, which were consistent between the two embodiments.

The term “computer” is intended to have a broad meaning that may be used in computing devices such as, e.g., but not limited to, standalone or client or server devices. The computer may be, e.g., (but not limited to) a personal computer (PC) system running an operating system such as, e.g., (but not limited to) MICROSOFT® WINDOWS® NT/98/2000/XP/Vista/Windows 7/8/etc. available from MICROSOFT® Corporation of Redmond, Wash., U.S.A. or an Apple computer executing MACR OS from Apple® of Cupertino, Calif., U.S.A. However, the invention is not limited to these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example, (but is not limited to) a hard disk drive and/or a removable storage drive, representing a floppy diskette drive, a magnetic tape drive, an optical disk drive, a compact disk drive CD-ROM, flash memory, etc. The removable storage drive may, e.g., but is not limited to, read from and/or write to a removable storage unit in a well-known manner. The removable storage unit, also called a program storage device or a computer program product, may represent, e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In alternative illustrative embodiments, the secondary memory may include other similar devices for allowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

The computer may also include an input device may include any mechanism or combination of mechanisms that may permit information to be input into the computer system from, e.g., a user. The input device may include logic configured to receive information for the computer system from, e.g. a user. Examples of the input device may include, e.g., but not limited to, a mouse, pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or other camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with processor either wired or wirelessly. A communications interface may allow software and data to be transferred between the computer system and external devices.

The term “data processor” is intended to have a broad meaning that includes one or more processors, such as, e.g., but not limited to, local processors or processors that are connected to a communication infrastructure (e.g., but not limited to, a communications bus, cross-over bar, interconnect, or network, etc.). The term data processor may include any type of processor, microprocessor and/or processing logic that may interpret and execute instructions (e.g., for example, a field programmable gate array (FPGA)). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. The data processor may be onboard, external to other components, or both. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CAT5, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

### Supplemental Materials

### Water Phantom Validation

A water phantom at room temperature demonstrated the impact of the residual CSF signal on the observed T₁-PALAN ΔS signal. The residual CSF is defined as the CSF signal that cannot be nulled due to individual differences or CSF inhomogeneity. The distilled water was placed in 5 mm NMR tubes for the MRI study. The MRI was performed using identical T₁-PALAN pulse sequence and MRI coils used in the mouse brain studies. Distilled water and CSF had slightly different T₁ and TIₙᵤₗₗ values. The TIₙᵤₗₗ of distilled water was determined to be 1.8 s by examining the water signal with respect to PLD.

FIG. 7A shows the simulated T₁-PALAN ΔS signal as a function of PLD for different residue CSF signals. FIGS. 7B, 7C, and 7D show the averaged T₁-PALAN ΔS signal of water phantom (n=3) as a function of PLD for different TI values. The observed signals ΔS of the T₁-PALAN embodiment as a function of PLD were simulated with different residual CSF values (−0.5, −0.01, 0.01, and 0.5), R_{1CSF}=0.33 s⁻¹ and Eq. 4. (FIG. 7A) For T1<TIₙᵤₗₗ, the residual signals were negative, while the residual signals were positive for TI>TIₙᵤₗₗ. For the residual CSF signal at 0.5, the ΔS signal peaked at PLD=0.9 s and then decayed with T_{1CSF}.

The averaged T₁-PALAN ΔS signals (n=3) as a function of PLD for water phantom are plotted for different TI values (1.6 s, 1.8 s, and 2.0 s) in FIGS. 7B-7D: TI=1.6 s (FIG. 7B), TI=1.8 s (FIG. 7C) and TI=2.0 s (FIG. 7D). With TI=1.6 s, the \S signal decreased and reached the minimum at 0.2 s (ΔS=−0.095±0.24), while the ΔS signal increased with TI=2 s and peaked at 0.2 s (ΔS=0.128±0.25). The two curves with TI=1.6 (FIG. 7B) and 2 s (FIG. 7D) closely resemble the simulations in FIG. 7A. With TIₙᵤₗₗ=1.8 s, water null time, the ΔS signals were less than 0.003 for PLD>0), indicating a well cancelation with control and label images in the T₁-PALAN embodiment.

### In Vivo Inversion Time Optimization

FIGS. 8A, 8B, and 8C show the averaged T₁-PALAN ΔS signal of mouse ventricles (third and lateral ventricle) (n=3) as a function of PLD for different TI values.

Due to the Rician noise, the residual CSF signal is hard to be extracted directly from the TI-dependent images (FIG. 2). The impact of the residual CSF signal on the mouse ventricle T₁-PALAN ΔS signal was demonstrated by performing multi-PLD T₁-PALAN with different TI values, i.e., TI=1.8 s (FIG. 8A), 2.0 s (FIG. 8B) and 2.2 s (FIG. 8C).

The observed multi-PLD T₁-PALAN signals for short T₁ (1.8 s) and long TI (2.2 s) followed the simulation curves (FIG. 7A) and the water phantoms curves (FIGS. 7B and 7D). It is a challenge to differentiate the residual CSF signal and the water exchange signal, of which is the sum of the ΔS. We used the signal at 0.1 s from the T₁-PALAN study to estimate the residual CSF signal because, at 0.1 s, the residual CSF signal dominated as shown in FIGS. 7A-7D, and the ΔS signal introduced by water exchange is still minimal. With TI=2 s at 0.1 s (FIG. 8B), the total signal is 0.0037, which is low (<11%) compared to the peak signal ΔS=0.034±0.01. As a comparison, the signals at 0.1 s for TI=1.8 s and 2.2 s are −0.031 and 0.042, respectively. In summary, the mouse CSF in ventricles was substantially suppressed with TI=2s for the T₁-PALAN embodiment, and the ΔS signal was introduced by water exchange in the brain. (FIGS. 7C and 8B).

In the real experiments, the inversion time (TI) was determined for each mouse with an uncertainty less than 10 ms. Also, the TI values of the CSF can vary slightly across different animal. The impact of imperfect T₁ and T₁ on the residual CSF signal was simulated with Eqs. 2-3, and the results are plotted in FIG. 9. FIG. 9 shows the simulated residual CSF signal with respect to the T₁ variation (ΔT₁) and T1 difference (ΔT₁). In the simulation, T₁=3.6 s and TI=2.12 s was used. The residual signal is less than 0.006 with ΔTI=10 ms with a perfect T₁ value. The maximum residual CSF (0.12) is found with ΔTI=10 ms/ΔT₁=−10 ms or with ΔTI=−10 ms/ΔT₁=10 ms.

### ADC Measurement on Mouse Brain

FIG. 10 shows the CSF and parenchyma MRI signals as a function of b values with the PGSE module. All the signals were normalized to the signal at b=0. Solid lines are fitting curves with Eq. 8.

The ADC values of CSF and parenchyma in the mouse brain were measured with twice-refocused pulsed gradient spin echo (PGSE) module. Parameters are as described above in the MRI Pulse Sequences section. In the ADC-PALAN embodiment:

$$S = A {e}^{- ADC \cdot b} + R , \qquad ( 8 )$$

with fixed echo time determined CSF ADC values as a function of the b value. R is the magnitude Rician noise background. Fitting with Eq. 8, ADC values were found to be 3.8=0.8 μm²/ms (CSF) and 0.9±0.3 μm²/ms (parenchyma water).

### T₂-PALAN Measurement on Mouse Brain

In principle, the measurement of CSF exchange with parenchyma can be achieved with the T₂-PALAN embodiment. In order to demonstrate the feasibility of T₂-PALAN embodiment, a Carr-Purcell-Meiboom-Gill (CPMG) module was used in the T₂-PALAN to null the parenchyma water signal. (see FIG. 7A and FIGS. 5A-5J). The CSF and parenchyma water exchange modulated the recovery curve of parenchyma. The label and control images were collected by alternating the phase of the second 90-degree pulse at the end of the CPMG module by 180-degrees. TSE with short TE (TE=5 ms) was used to acquire parenchyma MRI images with a pre-scan delay of 5 s, TSE factor=16, slice thickness=1.5 mm, FOV=1.6×1.6 mm². A matrix size of 32×32 was used for the CSF and parenchyma water change kinetic curve measurement with four pairs of control/label images. 9 PLDs (0, 0.5, 1, 1.5, 2, 2.5, 3, 4, and 6 s) were acquired for the CSF labeling efficiency measurement and T₂-PALAN kinetic curve measurement (n=3). In the T₂-PALAN multi-PLD studies, a single axis slice was collected at −0.7 mm from AC for the CSF optimization, and the slice at −4.1 mm from AC was used for the parenchyma signal optimization.

The T₂-PALAN measurement on mouse brain is presented in FIGS. 5A-5D. Differently from the ADC- and T₁-PALAN embodiments, the CSF signal was maintained and labeled in the T₂-PALAN sequence. The CSF recovery difference between the post-labeling and the original curves gives the labeling efficiency of 0.28±0.3 (FIGS. 5A and 5B). The parenchyma recovery curves after the label/control pulse (FIG. 5C) shows a barely noticeable difference, which indicates a nominal exchange rate between CSF and the parenchyma. The T₂-PALAN ΔS signal is under 0.22%. (FIG. 5D). Which such extremely low signal, it is very challenge to reliably extract the water exchange rates between CSF and parenchyma.

Further aspects of the present disclosure are provided by the subject matter of the following clauses.

A non-invasive MRI method for quantifying the interstitial fluid (ISF) and cerebrospinal fluid (CSF) flow, the CSF to ISF flow, and ISF between white matter (WM) and gray matter (GM) in brain, the method including generating a spatial map of water magnetic resonance (MR) signals that are sensitized to changes in the ISF to CSF flow, CSF to ISF flow and ISF flow among WM and GM in the cerebrospinal fluid (CSF) or parenchyma of the subject. The method further includes observing two spatial maps of subject brain ventricles, i.e., control images and label images, by alternating the phase of pulse in the T₁ or diffusion preparation modules on the subject ISF. The method further includes observing two spatial maps of subject brain parenchyma, i.e., control images and label images, by alternating the phase of pulse in the T₂ preparation modules on the subject CSF. The method further includes detecting a difference between the MR signals of the label and control images. The method further includes observing two spatial maps of subject brain parenchyma, i.e., control images and label images, by alternating the phase of pulse in the T₁ preparation modules on the subject WM or GM. The method further includes detecting a difference between the MR signals of the label and control images. The method further includes ascertaining the ISF to CSF, CSF to ISF flow and ISF flow between WM and GM associated with the brain lymphatic or glymphatic system of the subject based on the detected difference.

The method of the preceding clause, wherein the sensitizing to the flow rate between ISF and CSF comprises applying one T₁ sensitive module and nulling the CSF signal at a proper inversion time delay (TIₙᵤₗₗ) while the parenchyma ISF signal is partially recovered due to the different T₁ from CSF.

The method of any preceding clause, wherein one or more high-power pulses comprise adiabatic or hard excitation pulses used for inverting the water magnetization and one time delay (TI) after the inversion pulse.

The method of any preceding clause, wherein the applying two or more RF pulses at the null time point (TIₙᵤₗₗ) is such that the brain parenchyma ISF signal will be maintained at Z-direction as a control image.

The method of any preceding clause, wherein the applying two or more RF pulses at the null time point (TIₙᵤₗₗ) is such that the brain parenchyma ISF signal will be inverted or saturated by changing the pulse phases as a label image.

The method of any preceding clause, wherein a post-labeling delay periods (PLD) range from 0 to several seconds can be applied after the pulses at TIₙᵤₗₗ to determine the kinetic curves of the ISF-CSF flow process.

The method of any preceding clause, further comprising determining the flow rate from ISF to CSF from the amplitude difference between the control and label images for different PLD periods.

The method of any preceding clause, wherein the spin echo MRI sequence with a long echo time will be used to suppress the parenchyma signal, while maintain the CSF signal.

The method of any preceding clause, wherein the sensitizing to the flow rate between ISF and CSF comprises applying one diffusion sensitive module and nulling the CSF signal with high b values, while part of the parenchyma ISF is maintained due to much smaller apparent diffusion coefficient (ADC).

The method of any preceding clause, wherein a train of high-power pulses comprise adiabatic or hard excitation pulses used combined with gradient for detecting the diffusion weighted MRI signal including but limited to pulsed gradient spin echo (PGSE), twice-refocused PGSE, improved motion-sensitized driven-equilibrium (iMSDE) and stimulated echo acquisition mode (STEAM).

The method of any preceding clause, wherein the last or first 90-degree pulse in the diffusion preparation module was set to maintain the brain parenchyma ISF signal in the Z-direction as one control image.

The method of any preceding clause, wherein the last or first 90-degree pulse in the diffusion preparation module was changed to invert or saturate the brain parenchyma ISF signal as one label image.

The method of any preceding clause, wherein a post-labeling delay periods (PLD) range from 0 to several seconds can be applied after the diffusion module to determine the kinetic curves of the ISF-CSF flow process.

The method of any preceding clause, further comprising determining the flow rate from ISF to CSF from the amplitude difference between the control and label images for different PLD periods.

The method of any preceding clause, wherein the spin echo MRI sequence with a long echo time will be used to suppress the parenchyma signal, while maintain the CSF signal.

The method of any preceding clause, wherein the sensitizing to the flow rate from CSF to ISF comprises applying one T2 relaxation sensitive module and nulling the parenchyma ISF signal with long echo time, while the majority of the CSF is maintained due to the much long CSF T2 value.

The method of any preceding clause, wherein a train of high-power pulses comprise adiabatic or hard excitation pulses used for detecting the T2 weighted MRI signal including but limited to spin echo and Carr-Purcell-Meiboom-Gill (CPMG) preparation modules.

The method of any preceding clause, wherein the last or first 90-degree pulse in the T2 preparation module was set to maintain the brain CSF signal in the Z-direction as one control image.

The method of any preceding clause, wherein the last or first 90-degree pulse in the T2 preparation module was changed to invert or saturate the brain CSF signal as one label image.

The method of any preceding clause, wherein a post-labeling delay periods (PLD) range from 0 to several seconds can be applied after the T2 module to determine the kinetic curves of the CSF to ISF flow process.

The method of any preceding clause, further comprising determining the flow rate from CSF to ISF from the amplitude difference between the control and label images for different PLD periods.

The method of any preceding clause, wherein the MRI sequences are used to map the parenchyma signal.

The method of any preceding clause, wherein the sensitizing to the ISF flow rate between WM and GM comprises applying one T1 sensitive module and nulling the WM/GM signal at a proper inversion time delay (TIₙᵤₗₗ) while the another component GM/WM is partially recovered due to the different T1 between WM and GM.

The method of any preceding clause, wherein one or more high-power pulses comprise adiabatic or hard excitation pulses used for inverting the water magnetization and one time delay (TI) after the inversion pulse.

The method of any preceding clause, wherein the applying two or more RF pulses at the null time point (TIₙᵤₗₗ) is such that the brain WM or GM signal will be maintained at Z-direction as a control image.

The method of any preceding clause, wherein the applying two or more RF pulses at the null time point (TIₙᵤₗₗ) is such that the brain WM or GM signal will be inverted or saturated by changing the pulse phases as a label image.

The method of any preceding clause, wherein a post-labeling delay periods (PLD) range from ( ) to several seconds can be applied after the pulses at TIₙᵤₗₗ to determine the kinetic curves of the ISF flow process between GM and WM.

The method of any preceding clause, further comprising determining the flow rate from WM to GM or vice versa from the amplitude difference between the control and label images for different PLD periods.

The method of any preceding clause, wherein the any MRI sequence that can detect WM and GM regions stimulatingly can be used.

A computer readable medium programmed with elements comprising applying radiofrequency pulses with different time delays and phases, applying the radiofrequency pulses interleaved with gradients, analyzing the difference in magnetization change as a function of post-labeling delay, and generating images of the differences in magnetization change as a function of post-labeling delay.

The terms “light” and “optical” are intended to have broad meanings that can include both visible regions of the electromagnetic spectrum as well as other regions, such as, but not limited to, infrared and ultraviolet light and optical imaging, for example, of such light.

The terms “computer”, “server”, “processor”, and “memory” all refer to electronic or other technological devices. These terms exclude people or groups of people. As used in this specification, the terms “computer readable medium,” “computer readable media.” and “machine readable medium,” etc. are entirely restricted to tangible, physical objects that store information in a form that is readable by a computer. These terms exclude any wireless signals, wired download signals, and any other ephemeral signals.

The term “computer” is intended to have a broad meaning that may be used in computing devices such as, e.g., but not limited to, standalone or client or server devices. The computer may be, e.g., (but not limited to) a personal computer (PC) system running an operating system such as, e.g., (but not limited to) MICROSOFT® WINDOWS® available from MICROSOFT® Corporation of Redmond, Wash., U.S.A. or an Apple computer executing MACR: OS from Apple R of Cupertino, Calif., U.S.A. However, the invention is not limited to these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example, (but not limited to) a hard disk drive and/or a removable storage drive, representing a floppy diskette drive, a magnetic tape drive, an optical disk drive, a read-only compact disk (CD-ROM), digital versatile discs (DVDs), flash memory (e.g., SD cards, mini-SD cards, micro-SD cards, etc.), read-only and recordable Blu-Ray R discs, etc. The removable storage drive may, e.g., but is not limited to, read from and/or write to a removable storage unit in a well-known manner. The removable storage unit, also called a program storage device or a computer program product, may represent. e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In some embodiments, the secondary memory may include other similar devices for allowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

Some embodiments include electronic components, such as microprocessors, storage and memory that store computer program instructions in a machine-readable or computer-readable medium (alternatively referred to as computer-readable storage media, machine-readable media, or machine-readable storage media). The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

The computer may also include an input device may include any mechanism or combination of mechanisms that may permit information to be input into the computer system from, e.g., a user. The input device may include logic configured to receive information for the computer system from, e.g., a user. Examples of the input device may include, e.g., but not limited to, a mouse, pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or another camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal display's (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with processor either wired or wirelessly. A communications interface may allow software and data to be transferred between the computer system and external devices.

The term “data processor” is intended to have a broad meaning that includes one or more processors, such as, e.g., but not limited to, that are connected to a communication infrastructure (e.g., but not limited to, a communications bus, cross-over bar, interconnect, or network, etc.). The term data processor may include any type of processor, microprocessor and/or processing logic that may interpret and execute instructions, including application-specific integrated circuits (ASICs) and field-programmable gate arrays (FPGAs). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CAT5, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

The term “network” is intended to include any communication network, including a local area network (“LAN”), a wide area network (“WAN”), an Intranet, or a network of networks, such as the Internet.

The term “software” is meant to include firmware residing in read-only memory or applications stored in magnetic storage, which can be read into memory for processing by a processor. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

### REFERENCES

1. Abate O, Bollo E, Lotti D, Bo S. Cytological, immunocytochemical and biochemical cerebrospinal fluid investigations in selected central nervous system disorders of dogs. Zentralbl Veterinarmed B 1998; 45 (2): 73-85.
2. Segal M B. Extracellular and cerebrospinal fluids. J Inherit Metab Dis 1993: 16 (4): 617-638.
3. Brodbelt A, Stoodley M. CSF pathways: a review. Br J Neurosurg 2007; 21 (5): 510-520.
4. Proulx S T. Cerebrospinal fluid outflow: a review of the historical and contemporary evidence for arachnoid villi, perineural routes, and dural lymphatics. Cell Mol Life Sci 2021; 78 (6): 2429-2457.
5. Johanson C E, Duncan J A, 3rd, Klinge P M, Brinker T, Stopa E G, Silverberg G D. Multiplicity of cerebrospinal fluid functions: New challenges in health and disease. Cerebrospinal Fluid Res 2008; 5:10.
6. Welch K, Friedman V. The cerebrospinal fluid valves. Brain 1960; 83:454-469.
7. Casaca-Carreira J, Temel Y, Hescham S A, Jahanshahi A. Transependymal Cerebrospinal Fluid Flow: Opportunity for Drug Delivery? Mol Neurobiol 2018; 55 (4): 2780-2788.
8. Sarnat H B, Flores-Sarnat L. Encyclopedia of the Neurological Sciences (Second Edition). Daroff R B, Aminoff M J, editors: Academic Press Inc; 2014.
9 Iliff J J, Wang M, Liao Y, Plogg B A, Peng W, Gundersen G A, Benveniste H, Vates G E, Deane R, Goldman S A, Nagelhus E A, Nedergaard M. A paravascular pathway facilitates CSF flow through the brain parenchyma and the clearance of interstitial solutes, including amyloid beta. Sci Transl Med 2012; 4 (147): 147ra111.
10. Iliff J J, Lee H, Yu M, Feng T, Logan J, Nedergaard M, Benveniste H. Brain-wide pathway for waste clearance captured by contrast-enhanced MRI. J Clin Invest 2013; 123 (3): 1299-1309.
11. Absinta M, Ha S K, Nair G, Sati P, Luciano N J, Palisoc M, Louveau A, Zaghloul K A, Pittaluga S, Kipnis J, Reich D S. Human and nonhuman primate meninges harbor lymphatic vessels that can be visualized noninvasively by MRI. Elife 2017;6.
12. Ahn J H, Cho H, Kim J H, Kim S H, Ham J S, Park I, Suh S H, Hong S P, Song J H, Hong Y K, Jeong Y, Park S H, Koh G Y. Meningeal lymphatic vessels at the skull base drain cerebrospinal fluid. Nature 2019; 572 (7767): 62-66.
13. Harrison I F, Siow B, Akilo A B, Evans P G, Ismail O, Ohene Y, Nahavandi P, Thomas D L, Lythgoe M F, Wells J A. Non-invasive imaging of CSF-mediated brain clearance pathways via assessment of perivascular fluid movement with diffusion tensor MRI. Elife 2018; 7.
14. Taoka T, Masutani Y, Kawai H, Nakane T, Matsuoka K, Yasuno F, Kishimoto T, Naganawa S. Evaluation of glymphatic system activity with the diffusion MR technique: diffusion tensor image analysis along the perivascular space (DTI-ALPS) in Alzheimer's disease cases. Jpn J Radiol 2017; 35 (4): 172-178.
15. Kuo P H, Stuehm C, Squire S, Johnson K. Meningeal Lymphatic Vessel Flow Runs Countercurrent to Venous Flow in the Superior Sagittal Sinus of the Human Brain. Tomography 2018; 4 (3): 99-104.
16. Da Mesquita S, Louveau A, Vaccari A, Smirnov I, Cornelison R C, Kingsmore K M, Contarino C, Onengut-Gumuscu S, Farber E, Raper D, Viar K E, Powell R D, Baker W, Dabhi N, Bai R, Cao R, Hu S, Rich S S, Munson J M, Lopes M B, Overall C C, Acton S T, Kipnis J. Functional aspects of meningeal lymphatics in ageing and Alzheimer's disease. Nature 2018; 560 (7717): 185-191.
17. Gaberel T, Gakuba C, Goulay R, Martinez De Lizarrondo S, Hanouz J L, Emery E, Touze E, Vivien D, Gauberti M. Impaired glymphatic perfusion after strokes revealed by contrast-enhanced MRI: a new target for fibrinolysis? Stroke 2014; 45 (10): 3092-3096.
18. Lee H, Mortensen K, Sanggaard S, Koch P, Brunner H, Quistorff B, Nedergaard M, Benveniste H. Quantitative Gd-DOTA uptake from cerebrospinal fluid into rat brain using 3D VFA-SPGR at 9.4T. Magn Reson Med 2018; 79 (3): 1568-1578.
19. Stanton E H, Persson N D A, Gomolka R S, Lilius T, Sigurethsson B, Lee H, Xavier A L R, Benveniste H, Nedergaard M, Mori Y. Mapping of CSF transport using high spatiotemporal resolution dynamic contrast-enhanced MRI in mice: Effect of anesthesia. Magn Reson Med 2021; 85 (6): 3326-3342.
20. Ringstad G, Valnes L M, Dale A M, Pripp A H, Vatnehol S S, Emblem K E, Mardal K A, Eide P K. Brain-wide glymphatic enhancement and clearance in humans assessed with MRI. JCI Insight 2018; 3 (13).
21. Eide P K, Ringstad G. MRI with intrathecal MRI gadolinium contrast medium administration: a possible method to assess glymphatic function in human brain. Acta Radiol Open 2015; 4 (11): 2058460115609635.
22. Ringstad G, Vatnehol S A S, Eide P K. Glymphatic M RI in idiopathic normal pressure hydrocephalus. Brain 2017; 140 (10): 2691-2705.
23. Chen L, Wei Z, Chan K W, Li Y, Suchal K, Bi S, Huang J, Xu X, Wong P C, Lu H, van Zijl P C, Li T, Xu J. D-Glucose uptake and clearance in the tauopathy Alzheimer's disease mouse brain detected by on-resonance variable delay multiple pulse MRI. J Cereb Blood Flow Metab 2021; 41:1013-1025.
24. Huang J, van Zijl P C M, Han X, Dong C M, Cheng G W Y, Tse K H, Knutsson L, Chen L, Lai J H C, Wu E X, Xu J, Chan K W Y. Altered d-glucose in brain parenchyma and cerebrospinal fluid of early Alzheimer's disease detected by dynamic glucose-enhanced MRI. Sci Adv 2020; 6 (20): eaba3884.
25. Ellingson B M, Kim E, Woodworth D C, Marques H, Boxerman J L, Safriel Y, Mckinstry R C, Bokstein F, Jain R, Chi T L, Sorensen A G, Gilbert M R, Barboriak D P. Diffusion M RI quality control and functional diffusion map results in ACRIN 6677/RTOG 0625: a multicenter, randomized, phase II trial of bevacizumab and chemotherapy in recurrent glioblastoma. Int J Oncol 2015; 46 (5): 1883-1892.
26. Kim S G. Quantification of relative cerebral blood flow change by flow-sensitive alternating inversion recovery (FAIR) technique: application to functional mapping. Magn Reson Med 1995; 34 (3): 293-301.
27. Parkes L M, Tofts P S. Improved accuracy of human cerebral blood perfusion measurements using arterial spin labeling: accounting for capillary water permeability. Magn Reson Med 2002; 48 (1): 27-41.
28. Li K L, Zhu X, Hylton N, Jahng G H, Weiner M W, Schuff N. Four-phase single-capillary stepwise model for kinetics in arterial spin labeling MRI. Magn Reson Med 2005; 53 (3): 511-518.
29. Carr J P, Buckley D L, Tessier J, Parker G J. What levels of precision are achievable for quantification of perfusion and capillary permeability surface area product using ASL? Magn Reson Med 2007; 58 (2): 281-289.
30. Gregori J, Schuff N, Kern R, Gunther M. T2-based arterial spin labeling measurements of blood to tissue water transfer in human brain. J Magn Reson Imaging 2013; 37 (2): 332-342.
31. Ohene Y, Harrison I F, Nahavandi P, Ismail O, Bird E V, Ottersen O P, Nagelhus E A, Thomas D L, Lythgoe M F, Wells J A. Non-invasive MRI of brain clearance pathways using multiple echo time arterial spin labelling: an aquaporin-4 study. Neuroimage 2019; 188:515-523.
32. Wells J A, Siow B, Lythgoe M F, Thomas D L. Measuring biexponential transverse relaxation of the ASL signal at 9.4 T to estimate arterial oxygen saturation and the time of exchange of labeled blood water into cortical brain tissue. J Cereb Blood Flow Metab 2013; 33 (2): 215-224.
33. Shao X, Ma S J, Casey M, D′Orazio L, Ringman J M, Wang D J J. Mapping water exchange across the blood-brain barrier using 3D diffusion-prepared arterial spin labeled perfusion MRI. Magn Reson Med 2019; 81 (5): 3065-3079.
34. Silva A C, Williams D S, Koretsky A P. Evidence for the exchange of arterial spin-labeled water with tissue water in rat brain from diffusion-sensitized measurements of perfusion. Magn Reson Med 1997; 38 (2): 232-237.
35. St Lawrence K S, Owen D, Wang D J. A two-stage approach for measuring vascular water exchange and arterial transit time by diffusion-weighted perfusion MRI. Magn Reson Med 2012; 67 (5): 1275-1284.
36. Dickie B R, Parker G J M, Parkes L M. Measuring water exchange across the blood-brain barrier using MRI. Prog Nucl Magn Reson Spectrosc 2020; 116:19-39.
37. Wang J N, Yarnykh V L, Yuan C. Enhanced Image Quality in Black-Blood MRI Using the Improved Motion-Sensitized Driven-Equilibrium (iMSDE) Sequence. J Magn Reson Imaging 2010; 31 (5): 1256-1263.
38. Mao J, Mareci T H, E.RAndrew. Experimental study of optimal selective 180° radiofrequency pulses. J Magn Reson 1988; 79 (1): 1-10.
39. Lein E S, Hawrylycz M J, Ao N, Ayres M, Bensinger A, Bernard A, Boe A F, Boguski M S, Brockway K S, Byrnes E J, Chen L, Chen L, Chen T M, Chin M C, Chong J, Crook B E, Czaplinska A, Dang C N, Datta S, Dee N R, Desaki A L, Desta T, Diep E, Dolbeare T A, Donelan M J, Dong H W, Dougherty J G, Duncan B J, Ebbert A J, Eichele G, Estin L K, Faber C, Facer B A, Fields R, Fischer S R, Fliss T P, Frensley C, Gates S N, Glattfelder K J, Halverson K R, Hart M R, Hohmann J G, Howell M P, Jeung D P, Johnson R A, Karr P T, Kawal R, Kidney J M, Knapik R H, Kuan C L, Lake J H, Laramee A R, Larsen K D, Lau C, Lemon T A, Liang A J, Liu Y, Luong L T, Michaels J, Morgan J J, Morgan R J, Mortrud M T, Mosqueda N F, Ng L L, Ng R, Orta G J, Overly C C, Pak T H, Parry S E, Pathak S D, Pearson O C, Puchalski R B, Riley Z L, Rockett H R, Rowland S A, Royall J J, Ruiz M J, Sarno N R, Schaffnit K, Shapovalova N V, Sivisay T, Slaughterbeck C R, Smith S C, Smith K A, Smith B I, Sodt A J, Stewart N N, Stumpf K R, Sunkin S M, Sutram M, Tam A, Teemer C D, Thaller C, Thompson C L, Varnam L R, Visel A, Whitlock R M, Wohnoutka P E, Wolkey C K, Wong V Y, Wood M, Yaylaoglu M B, Young R C, Youngstrom B L, Yuan X F, Zhang B, Zwingman T A, Jones A R. Genome-wide atlas of gene expression in the adult mouse brain. Nature 2007; 445 (7124): 168-176.
40. Lin Z, Jiang D, Liu D, Li Y, Uh J, Hou X, Pillai J J, Qin Q, Ge Y, Lu H. Noncontrast assessment of blood-brain barrier permeability to water: Shorter acquisition, test-retest reproducibility, and comparison with contrast-based method. Magn Reson Med 2021; 86 (1): 143-156.
41. Calamante F. Arterial input function in perfusion MRI: a comprehensive review. Progress in Nuclear Magnetic Resonance Spectroscopy 2013: DO: 10.1016/j.pnmrs.2013.1004.1002.
42. Wong E C, Buxton R B, Frank L R. A theoretical and experimental comparison of continuous and pulsed arterial spin labeling techniques for quantitative perfusion imaging. Magn Reson Med 1998; 40 (3): 348-355.
43. Qin Q. Huang A J, Hua J, Desmond J E, Stevens R D, van Zijl P C M. Three-dimensional whole-brain perfusion quantification using pseudo-continuous arterial spin labeling MRI at multiple post-labeling delays: accounting for both arterial transit time and impulse response function. NMR Biomed 2014; 27 (2): 116-128.
44. Boxerman J L, Bandettini P A, Kwong K K, Baker J R, Davis T L, Rosen B R, Weisskoff R M. The intravascular contribution to fMRI signal change: Monte Carlo modeling and diffusion-weighted studies in vivo. Magn Reson Med 1995; 34 (1): 4-10.
45. Jochimsen T H, Norris D G, Mildner T, Moller H E. Quantifying the intra- and extravascular contributions to spin-echo fMRI at 3 T. Magn Reson Med 2004: 52 (4); 724-732.
46. Evans P G, Sokolska M, Alves A, Harrison I F, Ohene Y, Nahavandi P, Ismail O, Miranda E, Lythgoe M F, Thomas D L, Wells J A. Non-Invasive M RI of Blood-Cerebrospinal Fluid Barrier Function. Nat Commun 2020; 11 (1): 2081.
47. Enzmann D R, Pelc N J. Normal flow patterns of intracranial and spinal cerebrospinal fluid defined with phase-contrast cine MR imaging. Radiology 1991; 178 (2): 467-474.
48. Nitz W R, Bradley W G, Jr., Watanabe A S, Lee R R, Burgoyne B, O'Sullivan R M, Herbst M D. Flow dynamics of cerebrospinal fluid: assessment with phase-contrast velocity MR imaging performed with retrospective cardiac gating. Radiology 1992; 183 (2): 395-405.
49. Vivas-Buitrago T, Lokossou A, Jusue-Torres I, Pinilla-Monsalve G, Blitz A M, Herzka D A, Robison J, Xu J, Guerrero-Cazares H, Mori S, Quinones-Hinojosa A, Baledent O, Rigamonti D. Aqueductal Cerebrospinal Fluid Stroke Volume Flow in a Rodent Model of Chronic Communicating Hydrocephalus: Establishing a Homogeneous Study Population for Cerebrospinal Fluid Dynamics Exploration. World Neurosurg 2019; 128:e1118-el125.
50. Liu G, Mestre H, Sweeney A M, Sun Q, Weikop P, Du T, Nedergaard M. Direct Measurement of Cerebrospinal Fluid Production in Mice. Cell Rep 2020; 33 (12): 108524.
51. Bowsher D. Pathways of absorption of protein from the cerebrospinal fluid: an autoradiographic study in the cat. Anat Rec 1957; 128 (1): 23-39.
52. Sahar A, Hochwald G M, Sadik A R, Ransohoff J. Cerebrospinal fluid absorption in animals with experimental obstructive hydrocephalus. Arch Neurol 1969; 21 (6): 638-644.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. Moreover, features described in connection with one embodiment may be used in conjunction with other embodiments, even if not explicitly stated above.

1. A system for magnetic resonance imaging of water exchange processes, comprising:
a primary magnet configured to provide a magnetic field over an imaging volume;
a magnetic gradient coil configured to generate a spatial encoding in the magnetic field;
a radiofrequency (RF) coil configured to:
acquire, from the imaging volume, at a plurality of time points, a plurality of water magnetic resonance signals, said plurality of water magnetic resonance signals comprising a first subset of signals that are labeled for a water exchange process and a second subset of signals that are not labeled; and
a data processor configured to:
generate, from the first subset of signals, a plurality of labeled images;
generate, from the second subset of signals, a plurality of control images corresponding to the plurality of labeled images; and
calculate one or more parameters to characterize a water exchange process in the imaging volume between a first water compartment and a second water compartment based on the labeled images and the corresponding control images.

2. The system of claim 1, wherein the RF coil is further configured to null water signals from the second water compartment at a particular time.

3. The system of claim 2, wherein the RF coil is configured to null water signals from the second water compartment by applying an inversion pulse at an interval prior to the particular time, said interval being determined based on a T1 relaxation time constant associated with the second water compartment.

4. The system of claim 2, wherein the RF coil is configured to null water signals from the second water compartment by applying a diffusion pulse with at least one b-value prior to the particular time, said b-value being determined based on an apparent diffusion coefficient (ADC) of the second water compartment.

5. The system of claim 2, wherein the RF coil is configured to null water signals from the second water compartment by applying a Carr-Purcell-Meiboom-Gill (CPMG) pulse with an echo time ending at the particular time, said echo time being determined based on a T2 relaxation time constant associated with the second water compartment.

6. The system of claim 2, wherein the RF coil is further configured to acquire the first subset of signals by applying, at the particular time, a first 90-degree pulse and a second 90-degree pulse, wherein the second 90-degree pulse has an opposite phase from the first 90-degree pulse.

7. The system of claim 2, wherein the RF coil is further configured to acquire the second subset of signals by applying, at the particular time, a first 90-degree pulse and a second 90-degree pulse, wherein the second 90-degree pulse has a same phase as the first 90-degree pulse.

8. The system of claim 2, wherein the plurality of time points are subsequent to the particular time.

9. The system of claim 8, wherein the water magnetic resonance signals are acquired at the plurality of time points using a pulse sequence that suppresses water signals from the first water compartment to provide improved contrast for water signals from the second water compartment.

10. The system of claim 2, wherein the first water compartment is interstitial fluid (ISF) and ependyma, the second water compartment is cerebrospinal fluid (CSF), and the water exchange process is a flow from ISF and ependyma to CSF.

11. The system of claim 2, wherein the second water compartment is interstitial fluid (ISF), the first water compartment is cerebrospinal fluid (CSF), and the water exchange process is a flow from CSF to ISF.

12. The system of claim 2, wherein the second water compartment is white matter (WM), the first water compartment is gray matter (GM), and the water exchange process is a flow from GM to WM.

13. The system of claim 2, wherein the second water compartment is gray matter (GM), the first water compartment is white matter (WM), and the water exchange process is a flow from WM to GM.

14. The system of claim 1, wherein the parameters to characterize the water exchange process comprise at least one of a water exchange transit time expressed in units of time, and a flow rate expressed in units of volume per mass per unit time.

15. The system of claim 1, wherein the parameters to characterize the water exchange process are calculated using a difference of the labeled images and the corresponding control images.

16. A method for magnetic resonance imaging of water exchange processes, comprising:
receiving a plurality of water magnetic resonance signals that were acquired by a radiofrequency (RF) coil from an imaging volume at a plurality of time points, said plurality of water magnetic resonance signals comprising a first subset of signals that were labeled for a water exchange process and a second subset of signals that were not labeled;
generating, from the first subset of signals, a plurality of labeled images;
generating, from the second subset of signals, a plurality of control images corresponding to the plurality of labeled images; and
calculating one or more parameters to characterize a water exchange process in the imaging volume between a first water compartment and a second water compartment based on the labeled images and the corresponding control images.

17. The method of claim 16, wherein water signals from the second water compartment were nulled by the RF coil at a particular time.

18. The method of claim 17, wherein water signals from the second water compartment were nulled by the RF coil by applying an inversion pulse at an interval prior to the particular time, said interval being determined based on a T1 relaxation time constant associated with the second water compartment.

19. The method of claim 17, wherein water signals from the second water compartment were nulled by the RF coil by applying a diffusion pulse with at least one b-value prior to the particular time, said b-value being determined based on an apparent diffusion coefficient (ADC) of the second water compartment.

20. The method of claim 17, wherein water signals from the second water compartment were nulled by the RF coil by applying a Carr-Purcell-Meiboom-Gill (CPMG) pulse with an echo time ending at the particular time, said echo time being determined based on a T2 relaxation time constant associated with the second water compartment.

21.-30. (canceled)
